(19) United States
(12) Patent Application Publication
(10) Pub. No.: US 2019/0338349 A1
(43) Pub. Date: Nov. 7, 2019

(54) METHODS AND SYSTEMS FOR HIGH FIDELITY SEQUENCING

(71) Applicant: GRAIL, Inc., Mento Park, CA (US)

(72) Inventors: Oliver Claude Venn, San Francisco, CA (US); Alexander Tilo Dilthey, Bethesda, MD (US)

(21) Appl. No.: 16/071,244

(22) PCT Filed: Jan. 22, 2017
(86) PCT No.: PCT/US2017/014426

§ 371 Date: Jul. 19, 2018

(51) Int. Cl. C12Q 1/6869; C12Q 1/6827; G16B 20/20; G16B 30/10; G16B 5/20

(52) U.S. Cl. CPC C12Q 1/6869; C12Q 1/6827; C12Q 2535/122; G16B 30/10; G16B 5/20; G16B 20/20

(57) ABSTRACT

Systems and methods for high fidelity sequencing and identification of rare mutations at dilute concentrations in a sample are described herein. In various aspects, the use of specialized library preparation techniques including adapter ligation conditions and hybrid capture enrichment panels are used along with controls to increase yield of sequence-ready molecules and identify and minimize contamination and errors. Systems and methods also relate to analyzing sequencing data to differentiate true variants from false positives using ensembles and a quasi-maximum likelihood model.

### CROSS REFERENCE TO RELATED APPLICATIONS

This application claims priority benefit of the filing date of U.S. Provisional Patent Application Ser. No. 62/286,110, filed on Jan. 22, 2016, the disclosure of which application is herein incorporated by reference in its entirety.

### FIELD OF THE INVENTION

This invention relates to systems and methods for high fidelity sequencing and identification of dilute variants in a sample through assay optimization and data analysis.

### BACKGROUND

Millions of cases of cancer are diagnosed and hundreds of thousands die each year in the United States alone. At the root of many diseases, including cancer, are genetic mutations or variants in an individual's DNA. In the case of cancer, these mutations can lead to abnormal cell growth which may be uncontrollable and result in death. Early detection of these diseases and the underlying mutations can be essential in successfully treating such diseases. Recent advancements allow for the isolation of tumor derived and other cell-free nucleic acids from blood and other bodily fluids. These developments permit less expensive, non-invasive examination and characterization of patient mutations. Unfortunately, mutations of interest, especially at early stages of disease development, often occur at frequencies less than standard sequencing error rates. The majority of cell-free nucleic acids include an individual's normal genomic sequence while a much smaller amount is of tumor origin presenting with tumor specific mutations. Modern sequencing and analysis techniques perform at an error rate of 1 error in every 1,000 positions read or 99.9% and are often inadequate to detect rare tumor variants in cell-free samples such as blood or plasma. The problem is that it becomes nearly impossible to discriminate between actual mutations and false positives introduced through errors in the sequencing process. Accordingly, the promise of early identification of disease specific mutations occurring at low frequencies goes unrealized and the benefits of early intervention are lost.

### SUMMARY

The invention relates to methods and systems for high fidelity sequencing and identification of rare nucleic acid variants. Systems and methods of the invention may be used to identify rare variants in cell-free nucleic acid samples such as tumor specific mutations among a sample comprising a normal genomic nucleic acid majority. Systems and methods of the invention allow for the confident identification of mutations occurring at frequencies below 1:10,000 in a sample. Identification of such rare variants results from optimization of several steps in the sequencing process followed by analysis of sequencing reads based on aligned read pairs referred to herein as ensembles.

Systems and methods of the invention may find applications outside of rare variant identification such as sequencing optimization for a desired level of performance or sensitivity. By using the invention to tailor sequencing procedures to a specific application, practitioners can avoid additional costs and time by only requiring the exact number of sequencing reads necessary for the particular application.

Aspects of the invention include methods for sequencing nucleic acid. Steps of the method may include obtaining sequencing reads of a nucleic acid, identifying an ensemble comprising two or more sequencing reads with shared start coordinates and read lengths, determining a number of sequenced molecules comprised by the ensemble, identifying a candidate variant in the ensemble, and determining a likelihood of the candidate variant being a true variant using a likelihood estimation model and the determined number of sequenced molecules. In certain embodiments, the step of obtaining sequencing reads may further comprise preparing a sequencing library from the nucleic acid, amplifying the sequencing library, and sequencing the sequencing library using next generation sequencing (NGS). In certain embodiments, adapters may be ligated to the nucleic acid under conditions configured to allow adapter stacking. The preparation of the sequencing library may comprise ligating adapters to the nucleic acid at a temperature of about 16 degrees Celsius using a reaction time of about 16 hours. The amplification step may comprise PCR amplification and methods of the invention may further comprise selecting an over-amplification factor and a PCR cycle number required to detect variants at a specified concentration in a sample using an in-silico model.

In various embodiments, methods of the invention include designing a hybrid capture panel to target a genomic region based on factors comprising, guanine-cytosine (GC) content, mutation frequency in a target population, and sequence uniqueness and capturing the amplified nucleic acid using the hybrid capture panel before the sequencing step. The capturing step may include using a first hybrid capture panel targeting a sense strand of a target loci and a second hybrid capture panel targeting an antisense strand of the target loci.

In certain embodiments, a synthetic nucleic acid control, also referred to as control sequence, control spike-in, or positive control, may be added to the nucleic acid before amplification of the sequencing library and error rate may then be determined using sequencing reads of the synthetic nucleic acid control. The synthetic nucleic acid control may comprise a known sequence having low diversity across a species from which the nucleic acid is derived and having a plurality of non-naturally occurring mismatches to the known sequence and, in certain embodiments, the plurality of non-naturally occurring mismatches can be 4. The synthetic nucleic acid control may include a guanine-cytosine (GC) content distribution that is representative of the target loci of the hybrid capture panel or may include a plurality of nucleic acids comprising varying overlaps with a pull down probe of the hybrid capture panel. Error rate or candidate variant frequency may be determined using sequencing reads of the synthetic nucleic acid control.

In various embodiments, the nucleic acid may comprise cell free nucleic acid or may be obtained from a tissue sample, where obtaining sequencing reads further comprises fragmenting the nucleic acid before the preparing step. Fragmentation may be generated using sonication or enzymatic cleavage.

Methods of the invention may include discarding the candidate variant if the candidate variant is not identified on both a sense and an antisense strand of the nucleic acid.

In certain aspects, the invention includes systems for identifying a nucleic acid variant. Systems include a processor coupled to a tangible, non-transient memory storying instructions that when executed by the processor cause the system to carry out various steps. Systems of the invention may be operable to identify an ensemble comprising two or more sequencing reads with shared start coordinates and read lengths, determine a number of sequenced molecules comprised by the ensemble, identify a candidate variant in the ensemble, and determine a likelihood of the candidate variant being a true variant using a likelihood estimation model and the determined number of sequenced molecules.

In certain embodiments, system so the invention may be operable to discard the candidate variant if the candidate variant is not identified on both a sense and an antisense strand of the nucleic acid. Systems of the invention may be further operable to determine a target genomic region for the two or more sequencing reads based on factors comprising, guanine-cytosine (GC) content, mutation frequency in a target population, and sequence uniqueness.

### BRIEF DESCRIPTION OF DRAWINGS

FIG. 1 provides a diagram of methods of the invention.

FIG. 2 illustrates sequencing compatible adapter ligation products including stacked adapters.

FIG. 3 illustrates PCR results of ligation products with stacked adapters.

FIG. 4 illustrates the distribution of molecule lengths of a prepared cell-free DNA library.

FIG. 5 illustrates the distribution of molecule length of a cell-free DNA library post PCR amplification using adapter specific primers.

FIG. 6 provides a diagram of a hybrid capture panel design process.

FIG. 7 illustrates a use of synthesized DNA controls to identify contamination of cell-free DNA samples.

FIG. 8 illustrates a computer system of the invention.

### DETAILED DESCRIPTION

Systems and methods of the invention generally relate to high fidelity sequencing and identification of rare nucleic acid variants using optimized sequencing techniques and sequencing read analysis. A necessary condition for the detection and accurate frequency estimation of low abundance mutations in a population of molecules (mutant allele proportion <5000⁻¹) is to maintain the proportion of derived alleles N_{d} (corresponding to somatic variants) to ancestral alleles Nₐ (corresponding to the germ-line genome) and DNA from other sources N_{?} throughout the sample preparation and library preparation process.

$$f = \frac{{N}_{d}}{{N}_{a} + {N}_{?} + {N}_{d}}$$

The proportion of derived alleles f can be decreased by depleting N_{d} through losses in the sequencing library construction process, or increasing the denominator through contamination. Accordingly, in order to identify mutations or variants present in cell-free DNA at low levels of concentration in a sample including cells, one must minimize contamination and minimize loss of molecules during library preparation. The present application presents systems and methods for achieving those goals as well as sequencing analysis techniques for differentiating true variants from false positives. By optimizing library preparation and sequencing steps, reducing sequencing errors, and including variant verification steps, systems and methods of the invention allow for identification of variants present in nucleic acid samples at ratios of 1:10,000 or lower. Identification of rare variants has numerous applications including the identification of tumor, cancer, or disease specific mutations in cell-free DNA made up predominantly of a patient's normal genomic DNA. Systems and methods of the invention leverage the lower error rates of high fidelity PCR enzymes compared to the error rates of next-generation NGS sequencing machines to increase sensitivity in identifying sequence variants by increasing the number of molecules to be sequenced through PCR amplification of the sample combined with post sequencing analysis to confirm validity of candidate variants.

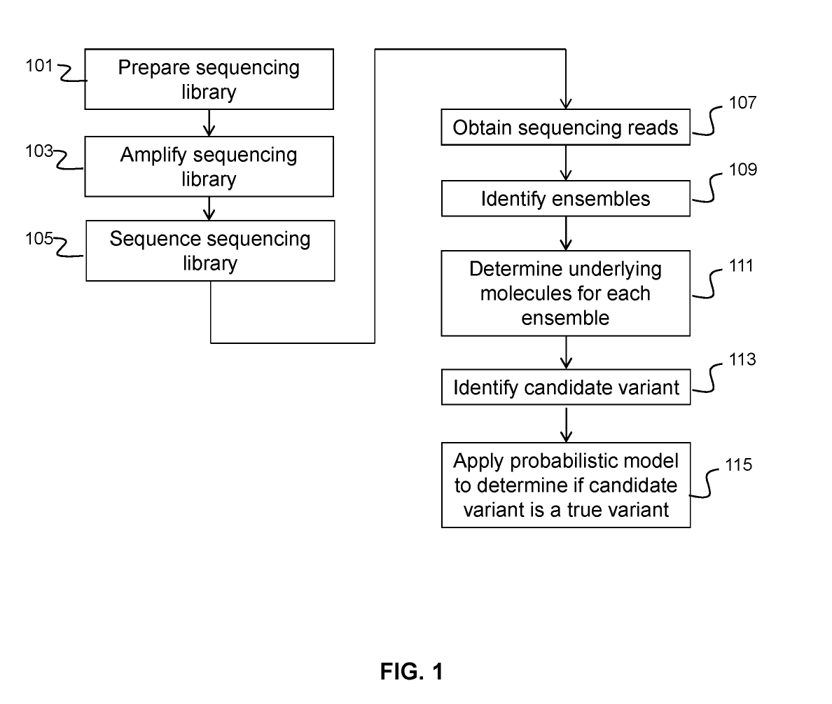

Systems and methods according to certain aspects of the invention are illustrated in FIG. 1. Steps may include sequencing library preparation 101, sequencing library amplification 103 and sequencing of the library 105. Systems and methods of the invention may be implemented by first obtaining sequencing reads 107 or may begin with a nucleic acid sample and the above steps to produce sequencing reads. Next, ensembles are identified in the sequencing reads 109 and the number of original molecules in the sample that underlie each ensemble are determined 111. Using the above information and a reference sequence, candidate variants are identified 113 and a probabilistic model is used to determine likelihood of a candidate variant being a true variant 115.

### Sample Preparation

In certain embodiments, nucleic acid may be obtained from a patient sample. Patient samples may, for example, comprise samples of blood, whole blood, blood plasma, tears, nipple aspirate, serum, stool, urine, saliva, circulating cells, tissue, biopsy samples, or other samples containing biological material of the patient. In preferred embodiments, nucleic acids are isolated from patient blood or plasma. Blood samples are processed quickly after being drawn to minimize contamination from DNA release by apoptotic nucleated cells.

An exemplary procedure for preparing nucleic acid from blood follows. Blood may be collected in 10 ml EDTA tubes (available, for example, from Becton Dickinson). Streck cfDNA tubes (Streck, Inc., Omaha, Nebr.) can be used to minimize contamination through chemical fixation of nucleated cells but little contamination from genomic DNA is observed when samples are processed within 2 hours or less as in preferred embodiments. Beginning with a blood sample, plasma may be extracted by centrifugation at 3000 rpm for 10 minutes at room temperature minus brake. Plasma may then be transferred to 1.5 ml tubes in 1 ml aliquots and centrifuged again at 7000 rpm for 10 minutes at room temperature. Supernatants can then be transferred to new 1.5 ml tubes. At this stage, samples can be stored at −80° C. In certain embodiments, samples can be stored at the plasma stage for later processing as plasma may be more stable than storing extracted cell-free (cfDNA).

Nucleic acid (e.g., DNA) can then be extracted from a blood sample (e.g., a blood plasma sample) using commercially available assays, for example, the Qiagen QIAmp Circulating Nucleic Acid kit (Qiagen N.V., Venlo Netherlands). In certain embodiments, the following modified elution strategy may be used. DNA may be extracted using the Qiagen QIAmp circulating nucleic acid kit following the manufacturer's instructions (maximum amount of plasma allowed per column is 5 ml). If cfDNA is being extracted from plasma where the blood was collected in Streck tubes, the reaction time with proteinase K may be doubled from 30 min to 60 min. Preferably, as large a volume as possible should be used (i.e. 5 mL). In various embodiments, a two-step elution may be used to maximize cfDNA yield. First DNA can be eluted using 30 μl of buffer AVE for each column. A minimal amount of buffer necessary to completely cover the membrane can be used in elution in order to increase cfDNA concentration. By decreasing dilution with a small amount of buffer, downstream desiccation of samples can be avoided to prevent melting of double stranded DNA or material loss.

Subsequently about 30 μl of buffer for each column can be eluted. In preferred embodiments, a second elution may be used to increase DNA yield. Table 1 shows the amounts of DNA observed cfDNA samples from six melanoma patients using a first and second elution in the above method where both elution volumes were about 30 μl. The usefulness of additional elutions may be determined by balancing the additional DNA obtained against decreasing the final DNA concentration in the elution. The elutions may then be combined and DNA quantified, preferably in triplicate, using commercially available assays such as the Qubit DNA high sensitivity kit (Thermo Fisher Scientific, Inc., Cambridge, Mass.).

TABLE 1
DNA concentrations in elutions

| | Plasma | | |
|---|---|---|---|
| | volume | Elution 1 | |
| Sample ID | (mL) | (ng) | Elution 2 (ng) |
| Plasma 009 | 3 | 12.63 | 5.22 |
| Plasma 010 | 3 | 11.76 | 6.12 |
| Plasma 045 | 3 | 21 | 4.14 |
| Plasma 020 | 3 | 20.94 | 5.7 |
| Plasma 062 | 3 | 17.1 | 5.88 |
| Plasma 063 | 3 | 18.9 | 6.6 |

### Library Preparation

While standard library preparation can be used to generate libraries, high yield protocols of the invention improve performance over standard approaches that often have library conversion yields of about 40%. Methods of the invention provide library conversion of about 80%. According to the invention, a sequencing library may be prepared from the nucleic acid sample. Commercially available kits may be used to prepare the sequencing library, such as Illumina's TruSeq Nano kit (Illumina, Inc., San Diego, Calif.) for whole genome sequencing (WGS). The reagent stoichiometry and incubation times may be modified to increase the number of molecules with correct sequencing adapter ligation through the process (library conversion efficiency). If the sample target is cfDNA in the sample, then no fragmentation is needed. In certain embodiments, nucleic acids may be obtained from tissue samples such as a tumor biopsy. In such instances, nucleic acids should be fragmented using means known in the art such as sonication or enzyme restriction. In practice, the average length of an unfragmented cfDNA population may be about 150-180 bases and varies from individual to individual. No solid phase reversible immobilization (SPRI) bead cleanup steps are used in preferred embodiments, instead, samples are taken straight to end repair to minimize loss of cfDNA. This eliminates the risk of ethanol carry over into PCR; ethanol is an inhibitor of PCR and it is challenging to remove all Ethanol droplets before SPRI beads start to crack. Avoiding the SPRI cleanup step additionally reduces operation time and cost. Reagent volumes may be adjusted by factor A based on the estimated number of DNA fragments in the sample to account for the different number of cfDNA fragments N_{f} relative to the fragments from sonicated genomic DNA N_{g} specified in TruSeq Nano protocol. This adjustment may be applied to reagents used in End Repair, 3′ End Adenylation, and Adapter Ligation steps. The number Nᵢ of molecules in population i can be calculated by dividing the mass of the population mᵢ by the product of the average molecular weight of one dideoxyribonucleotide (w=6.5E+11 ng/mole) and the average number of bases in each molecule Lᵢ, then multiplying this value by Avogadro's constant as follows:

$${N}_{i} = \frac{{m}_{i}}{w \times {L}_{i}} \times {N}_{A} .$$

The adjustment factor A is then the quotient of N_{f} divided by N_{g}:

$$A = \frac{{m}_{f}}{{m}_{g}} \times \frac{{L}_{g}}{{L}_{f}}$$

In certain embodiments, m_{g}=100 ng of input DNA, and specified sonication to fragment length of L_{g}=350 bases and m_{f} and L_{f} may be determined experimentally for a given sample using the equation above. Nucleic acid samples may then be processed further using known end repair techniques to ensure that each molecule is free of overhangs, and contains 5′ phosphate and 3′ hydroxyl groups followed by 3′ adenylation and adapter ligation.

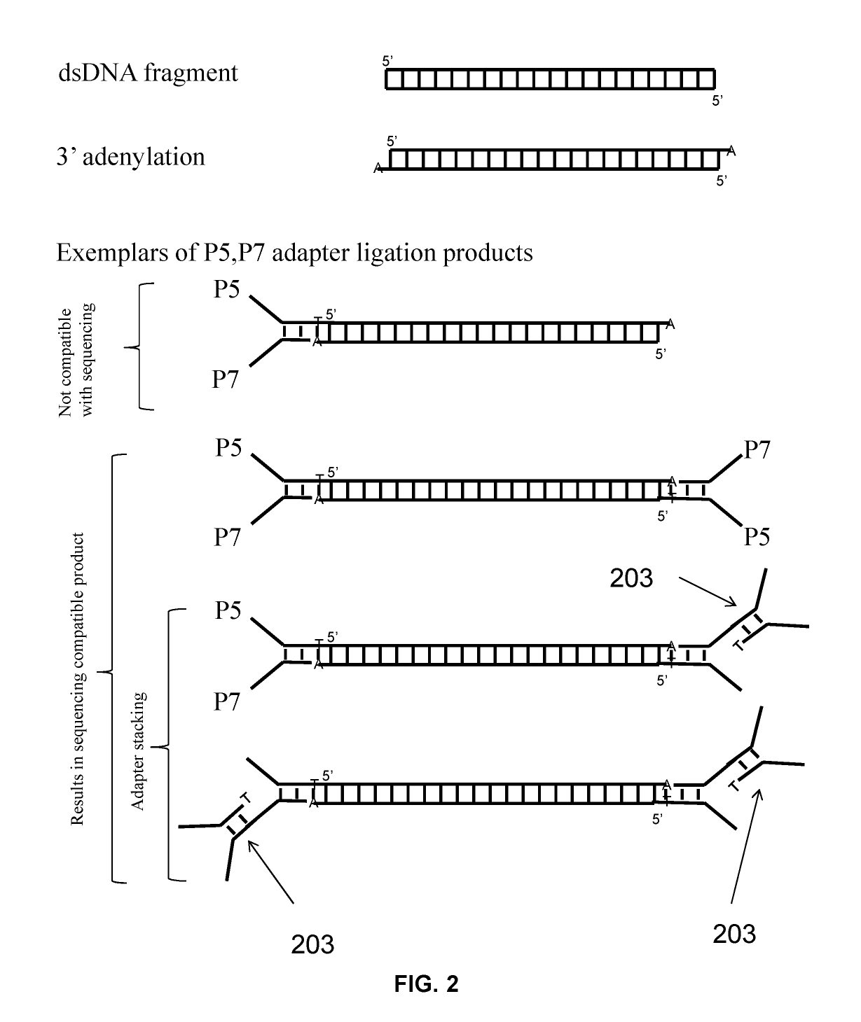

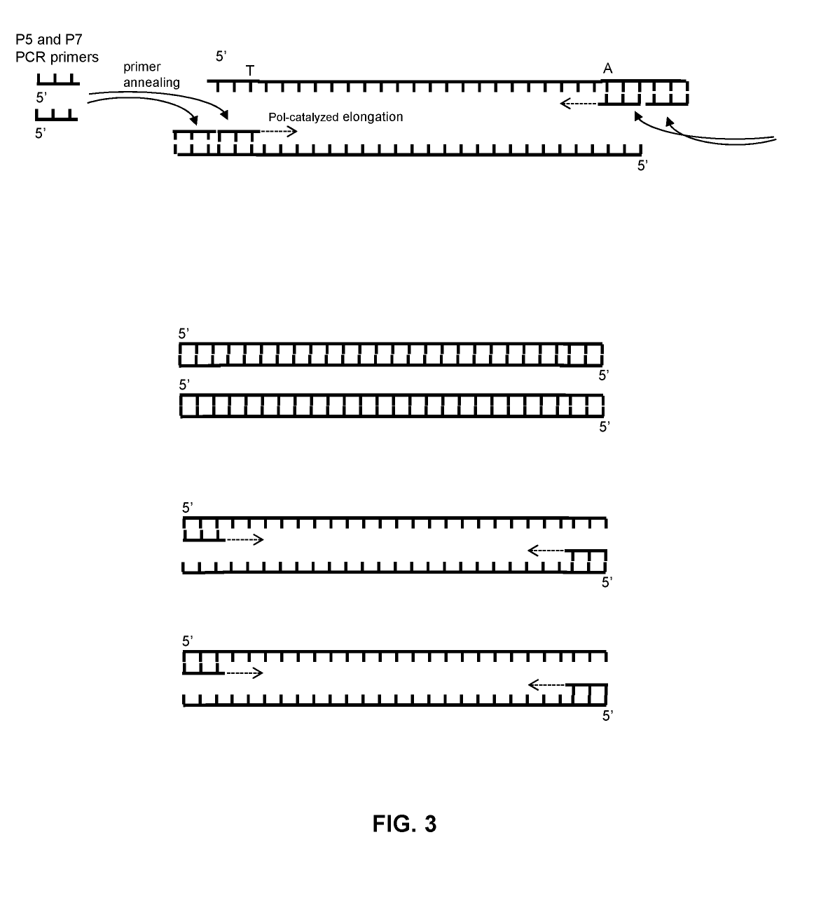

In various embodiments, a modified adapter ligation procedure can be used to increase yield of adapter ligated cfDNA fragments. To maximize the number of cfDNA fragments that have at least two Y-shaped Illumina sequencing adapters ligated (where Illumina sequencing is used), adapter ligation reaction time may be increased to 16 hours and/or the kinetic energy of the molecules in solution may be decreased using a lower incubation temperature of 16 C. In certain embodiments, adapter ligation may be performed under conditions, such as those just described, that encourage adapter ligation and can result in ‘stacking’ of adapters as shown in FIG. 2. (203). Stacked adapters, after PCR amplification resolve so that original molecules descendant PCR products are not prevented from being sequenced. FIG. 3 illustrates the resolution of stacked adapters during the PCR process. Steric hindrance results in the inner most primer being selected over the PCR cycles of amplification. Where the innermost primer binds prior to or at the same time as the outermost primer, the outermost primer site will be eliminated in the resulting PCR product. The time for the innermost primer to anneal before the outermost is geometrically distributed with a probability of success about 0.5 such that, after 4 rounds of PCR amplification, the probability of obtaining a sequencing compatible product is about 15:16.

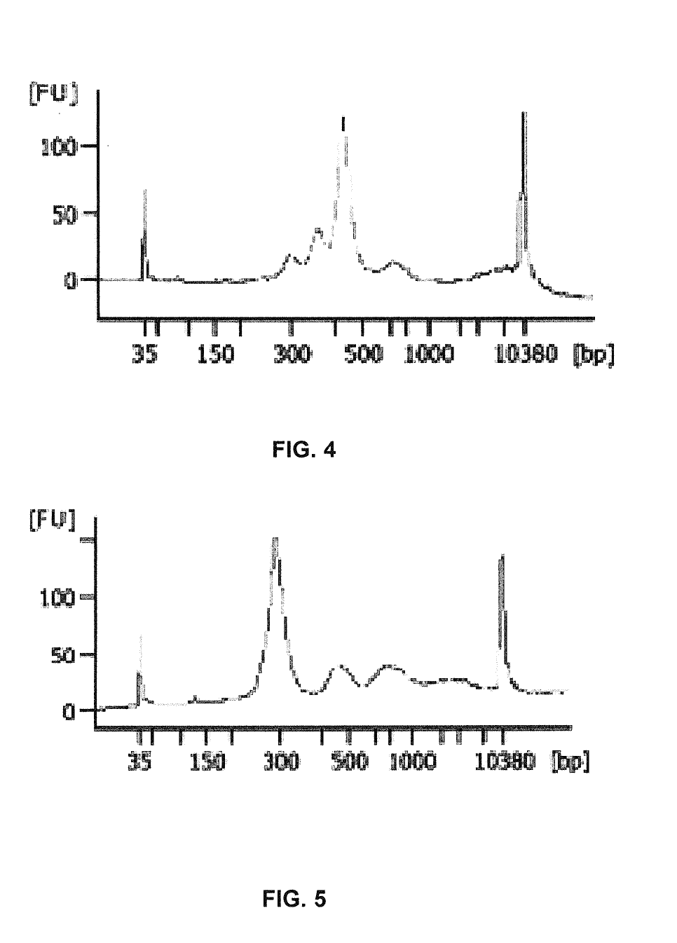

FIG. 4 illustrates the fragment length of a cfDNA library from a lung cancer patient where average molecule length is 174 bases and each adapter is 60 bases. FIG. 5 illustrates the prepared library after PCR amplification using adapter specific primers. These graphs illustrate that adapters stacking occurred and that the stacked adapters were effectively resolved through PCR amplification, resulting in a higher yield of molecules that are compatible with paired-end sequencing. The first three peaks in FIG. 4 correspond to the average molecule length plus 2, 3, and 4 adapters.

Amplified samples may then be cleaned up using SPRI sample purification beads at a ratio of 1:1.6 and then 1:1 of sample:beads in order to remove free adapters. Samples may then be eluted to a volume of about 27.5 μl.

According to certain embodiments, the sample fragment length can then be determined using, for example, a Bioanalyzer (Agilent Technologies, Santa Clara, Calif.) or equivalent instrument. About 1 μl of cfDNA may be input to identify average fragment length pre- and post-library preparation. The distribution of cfDNA molecule lengths prior to sequencing library preparation can be approximated as sampling from a Normal distribution, Xₚᵣₑ˜N (μₚᵣₑ, σ²), with mean length μ₀ about 150-180 bases, and sample variance σ². The distribution of molecule lengths post library preparation, Xₚₒₛₜ, is a superposition of Normal distributions shifted by the number of ligated sequencing adapters, each sequencing adapter has fixed length A, which is usually 60 bases for Illumina platforms described above (P5 and P7 adapters). Molecules that can be sequenced (sequencable) have at least 1 adapter ligated to each end of the cfDNA fragment, thus having a mean of μ₀+kA, where k≥2. If the library is PCR amplified, sequencable molecules may be generated if the number of ligated adapters, k, is at least 2:

$${X}_{post} \sim {\sum }_{k = 0}^{4} {Y}_{k} \times N ( ( {μ}_{pre} + kA ) , {σ}^{2} ) , k ∈ {ℕ}_{0}$$

Where Yₖ is the weight of the contribution of molecules with k adapters ligated. After PCR amplification using P5 and P7 PCR primers the total population should be dominated by μₚᵣₑ+2A (as shown in FIGS. 3 and 4).

The mass of the library may be quantified using a Kapa Library Quantification Kit (Kapa Biosystems, Inc. Wilmington, Mass.). The library may be amplified using any known amplification method including PCR amplification. In order to further reduce error rates, in preferred embodiments, library amplification may be conducted using Kapa HiFi Hotstart amplification (Kapa Biosystems, Inc. Wilmington, Mass. KR0370-v5.13). High fidelity PCR enzymes with robust performance across GC content such as Kapa HiFi Hotstart has up to 100× lower error rates than that of Taq polymerase. The level of duplicate reads may impact the total amount of required sequencing. A simulation engine can be used to assess the optimal over-amplication factor to detect variants at specified frequencies, jointly incorporating losses during library prep, induced errors, and calling algorithm dependencies. The simulation may account for losses in PCR amplification and hybrid capture or other pull-down or enrichment techniques where applicable.

The ratio of reads to underlying original molecules in an ensemble may be referred to as the Over-amplification Factor. To calculate the number of samples that can be analysed on one sequencing run, the following formula may be applied:

$$( \frac{samples}{run} ) = ⌊ ( \frac{reads}{run} ) \div \frac{\begin{matrix}( \frac{\# genome equivalents}{sample} ) \times \\ ( panel size ) \times ( overamplification factor )\end{matrix}}{average library molecule length} ⌋$$

This ensures efficient utilization of each sequencing run, while ensuring that there are enough reads for ensembles to be represented in the sequencing. The number of PCR cycles required to achieve desired redundancy can be calculated using a model fit to previous PCR runs. First PCR efficiency can be calculated by fitting exponential model to a known input amount of cfDNA. Then, using the estimated parameters the total number of amplifications required to achieve desired over-amplification can be calculated.

### Library Enrichment

In various embodiments, library enrichment may be used prior to sequencing in order to increase the likelihood that variants in targeted regions are identified. Enrichment may be through methods such as targeted PCR or hybrid capture panels. Targeted high throughput sequencing may be used to reduce the total number of sequencing reads required to assess specified loci in an individual. The reduction in required reads is a function of the quotient targeted sequence length divided by genome length, and weights determined by the distribution sequencing read depth of coverage (henceforth abbreviated as coverage) for the targeted and whole genome sequencing.

Increased coverage improves sensitivity since the number of reads containing a target allele is approximately binomially distributed with true variant proportion (1−ε)×f where ε is the base error rate in sequencing and f is the frequency of the allele in the molecule population and coverage D. Increased coverage can reduce false positives by enabling aggregating information across reads spanning a target locus (integrating out errors). More complicated error models are required because systematic error modes exist in sequencing, such as errors in homopolymers.

Selecting which regions of the genome to target is an important consideration in the design of targeted sequencing panels. In the context of cancer detection using genetic variant signatures, the statistical power of the targeted panel is a function of the recurrence of variants within the patient population across those loci. An additional consideration in hybrid capture design, is the specificity of each hybridization probe and the uniformity of sensitivity across all the probes, both drive the amount of sequencing reads required to detect variants at a desired limit of detection.

Systems and methods of the invention may focus on selecting a combination of loci up to a total sequence length L which optimizes for the greatest combined recurrence load in cancer patients (combining both driver and passenger genetic variants), accounting for determinants such as sequence uniqueness and GC content that affect hybrid capture performance. Additionally, the invention may use synthetic nucleic acid spike-ins that match cfDNA length distribution, and span the observed distribution of GC-content across target regions. The spike-ins are distinguishable from cfDNA based on specified reference mismatches, the pattern of mismatches was chosen such that they are unlikely to be observed from natural processes. These spike-ins are used to calculate estimates of false negative rate across GC contexts and predicted hybrid capture overlap.

Hybrid capture panels of the invention may be designed by identifying regions that are recurrently somatically mutated (focal amplifications, translocations, inversions, single nucleotide variants, insertions, deletions), and pre-specified loci (such as oncogene exons), and choosing the most informative combination of regions up to a specified total panel size. Hybrid capture panels may be designed with consideration of genome length, genomic alterations under consideration and forced inclusion of specified genes; tumor variation database under consideration and tumor types, and relative weights of each database; corrections for population incidence of each tumor type (to guard against sampling bias; and generation of target regions at exome, or genome level.

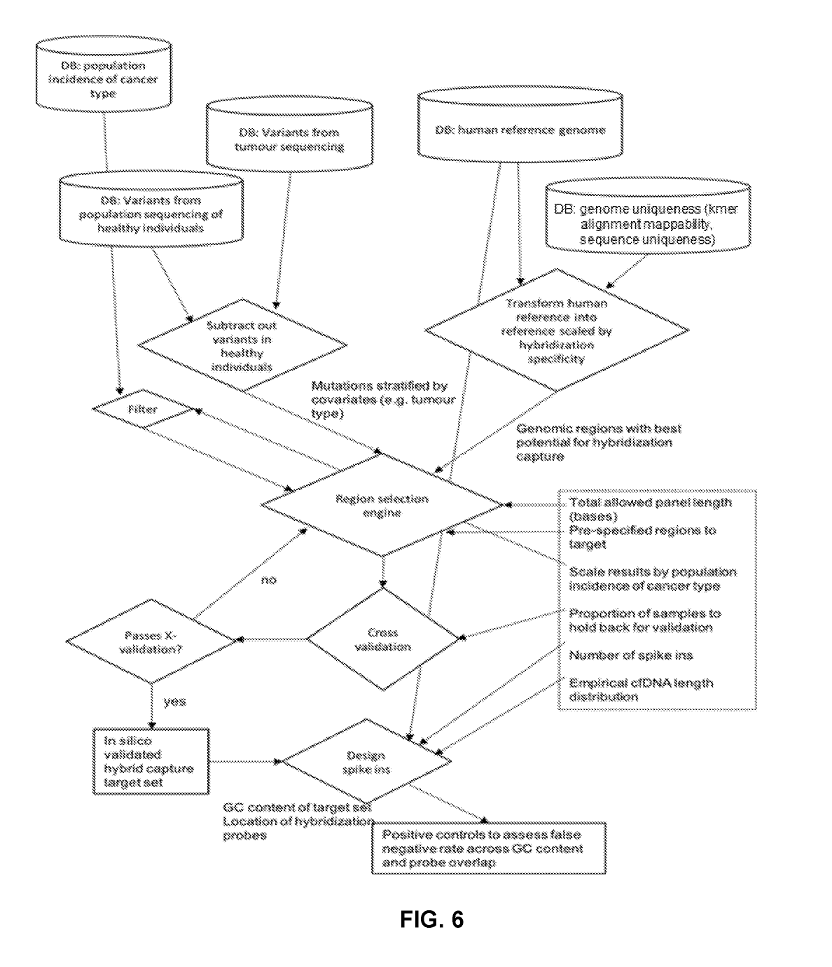

FIG. 6 provides a diagram of the hybrid capture panel design process according to certain embodiments including data transformations. Drums represent databases, dotted boxes represent inputs, diamonds represent operations, and solid border boxes represent outputs.

Inputs into the hybrid capture panel design process may include total allowed panel length in bases, pre-specified regions to target, weighting results by population incidence of cancer type, proportion of samples to hold back for validation, number of control spike-ins, and empirical nucleic acid length distribution.

Reference databases (DB) may include population incidence of the target cancer type, known variants from tumor sequencing, a human reference genome such as may be obtained from the genome reference consortium (http://www.ncbi.nlm.nih.gov/projects/genome/assembly/grc/human/), known variants from sequencing data of a healthy population, and genome uniqueness (e.g., kmer alignment mappability and sequence uniqueness). Databases may be determined experimentally and information may be added to the databases through application of the methods of the invention. Operations performed on the database information may include those operations designated within diamonds in FIG. 6. The outputs of the hybrid capture panel design may include the hybrid capture target set and positive controls to spike in to the sample or other wise use to assess false negative rate across guanine-cytosine (GC) content distribution.

To identify the most informative regions of the genome to target for a specified total panel length, the signatures of genetic alterations recorded in cancer mutation databases such as COSMIC (Catalogue of somatic mutations in cancer http://cancer.sanger.ac.uk/cosmic) may be assessed. Optimization may be carried out using either Forward-Backward optimization or Greedy Optimization.

Hybrid capture panel design may be validated using a cross validation procedure to account for potential biases induced by constructing the panels from a limited number of samples. Cross validation strategies can be important when designing cancer panels because the genetic variation in samples is heterogeneous both within tumors (intra-tumor heterogeneity) and between patients (inter-tumor heterogeneity), and are influenced by factors such as genetic background (e.g., POLE mutation status), environmental exposure (e.g., smoking history, previous therapy), and tumor stage.

For Forward Backward optimization, loci may be identified by alternating between forward and backward passes until a panel of specified length is constructed from L loci. Loci can be stratified into those included in the panel (chosen loci), and those not included on the panel (available loci). For each iteration, in the forward pass, the locus in the available loci which adds the greatest number of somatic mutations to the panel, f* can be identified. In the backward pass f* may be included into the panel set and the locus in the included loci that adds the least somatic recurrence, b* can be identified. If f* does not equal b*, b* can be excluded. The iterations can be repeated. This scheme may be used to identify an optimized set of loci for combined somatic recurrence. The optimization may end when the panel length is reached.

Using Greedy optimization, the process may start with the locus that adds the greatest somatic mutation load, add this to the panel, then choose from the remaining loci the locus with the greatest somatic mutation load. The process may terminate when the combined sequence meets the specified panel size.

Cross Fold Validation may be used to assess the stability of the identified panel accounting for the influence of structure in the disease databases.

In certain embodiments, two mutually exclusive sets of patient samples may be constructed, with the cardinality of the sets determined by the training proportion p. A panel can be generated on the first set that has cardinality p recording the total number of patients with mutations on the panel. The proposed panel can then be validated in the validation set that has cardinality (1−p), calculating the proportion of patients with mutations on the panel. If the patient proportions are within a threshold, T, the panel may be retained and may be revised if the proportions are not within T.

Databases of tumor biopsy sequencing may be queried to obtain samples of genetic variation, samples can be stratified by a number of patient covariates such as disease type, stage, environmental exposures, and histology. All germ line genetic variants observed in population sequencing of healthy populations can then be removed, such as the 1000 Genomes database, to guard against false positive variants in the cancer databases which would confound the panel design (this step is only useful where the target variants are disease related as in cancer diagnostics). There are known germline mutations, such as BRCA1/2 mutations that predispose individuals to cancer, which might be eliminated through such an approach but known regions of interest may be forced into the hybrid capture panel design to overcome these omissions if desired.

To account for differential performance in hybridization capture, information about the sequence properties of the human genome can be incorporated into the panel selection process. In certain embodiments metrics about the uniqueness of each base in the genome may be incorporated in the design process, since this drives the specificity of the hybrid capture. For example, if a locus is homologous (identical) to 99 other loci in the human genome (e.g., a LINE element), a capture probe would only pull down an average of 1 relevant locus per every 100. (The metrics used are 1).

This information may be incorporated by using two pre-calculated summary statistics of genome uniqueness available from the UCSC genome browser database (https://genome.ucsc.edu/).

Mappability, s, which quantifies the uniqueness of kmer sequence alignment to the genome

$$S = \frac{1}{( \# matches in the genome )}$$

Uniqueness, u, the uniqueness of 35 base windows across the genome in 1 base sliding windows

$$u ( x ) = \{ \begin{matrix}1 / x , & x < 4 \\ 0 , & x \geq 4\end{matrix} , where x is the number of exact shared sequences$$

The maps can be combined, and then a character encoded uniqueness value generated for each base in the human reference genome. The reference genome may thereby be transformed from a sequence of nucleotides, to a sequence of nucleotides annotated by a hybridization specificity score f (s, u).

Once a hybrid capture panel is designed, the panel may be used to enrich the sample for target genomic areas using their nucleotide sequence. To capture molecules, the double stranded DNA is melted into single stranded DNA (e.g., by increasing the temperature), then the hybrid capture probes (probes) are added, and conditions changed to encourage strand annealing. Probes are complementary to the target sequence and have a selectable marker (e.g., biotinylated) that enable the molecules to be isolated. To prevent hybridization between hybridization probes in a probe pool, all the probes in a pool are designed to all complement either the sense or anti-sense sequence of the target loci. Consequently, only one strand is captured per double stranded DNA molecule.

In certain embodiments, hybrid capture panels may be designed to specifically target both sense and anti-sense strands of DNA. Where the sample DNA is PCR amplified prior to hybridization capture, both strands of the original molecule are represented in the sense and anti-sense PCR duplicate population. For the purposes of clarity consider the following example: x={x₊, x₋} is a double stranded molecule, α and β are single stranded DNA molecules of length l, α shares complementary sequence for its first n consecutive bases with the last n consecutive bases of β, the remaining sequence is non-complementary. Consequently, α annealed to β has a forked Y-shaped structure of a double stranded DNA stem from the complementary sequence, and single stranded DNA arms from the non-complementary sequence.

Next, a molecule can be created where x is flanked on either end by the Y-shaped {α, β} double stranded DNA using known ligation reactions, e.g. blunt end ligation:

αx₋/β

βx₊α

PCR amplification may then be applied using primers complementary to α and β, represented as α_{c} and β_{c} respectively, to generate the family of PCR duplicates:

$$α {x}_{-} β {\to }^{PCR} \{ \begin{matrix}{α}_{c } {x}_{+} {β}_{c} , & α {x}_{-} β \}\end{matrix} \\ β {x}_{+} α {\to }^{PCR} \{ \begin{matrix}{β}_{c} {x}_{+} {α}_{c} , & β {x}_{+} α \}\end{matrix}$$

Applying a hybrid capture protocol with probe sequence x₋ to the PCR products, would extract molecules with sequence α_{c} x₊β_{c} and β x₊α, which each are descendants of the anti-sense and sense strands respectively. Subsequent PCR generates all four single stranded molecules classes, so both strands are represented. However, half of the molecules are not captured using this approach. Current capture methods only use one set of either sense or anti-sense probes however, strand specific isolation can be used to generate two identically distributed samples from the original sampled DNA. Such a method has utility for applications that seek to detect molecules at low frequency in a heterogeneous population as a means of controlling for errors and dropout induced in subsequent manipulation of the sampled DNA. For example, certain embodiments of the invention rely on candidate variants being represented on both sense and anti-sense strands for validation. In such methods, the strand specific isolation methods may be of particular use. Strand specific isolation may be achieved using the following steps.

Two hybrid capture panels may be created for the loci of interest; one sense (A), and one antisense (B). The panels may then be applied, in series, to the DNA sample. The selectable probes can be applied to single stranded DNA, separating the sample into the isolate (DNA bound by probes) partition and non-isolate partition (DNA not bound by probes) using standard hybrid capture protocols. Panel A can be applied to the DNA population. The target sequence will be collected in the isolate partition. The non-isolate partition may be retained. Panel B can then be applied to the non-isolate partition. The complement of panel 1 target sequence can be collected in the isolate partition of STEP 2.

There may be some carry over contamination of probes from A, but this should be minimal if isolation methods are optimized. In an alternative implementation, the sample may be partitioned into two aliquots and A and B treated separately, thereby avoiding any cross-hybridization that results from probe carry through in the previous step.

Isolates from A and B may be analyzed separately, then compared for concordance in the results between the two analyses, which controls for artifacts that are introduced in downstream treatment of the samples. This provides the opportunity for replication between isolates A and B, and increases sensitivity by assessing A and B separately.

### Sequencing

Samples may be diluted to 2 nM initially and then to a final concentration of 19 pM in 600 ul before sequencing. Suitable sequencing methods include, but are not limited to, sequencing by hybridization, SMRT™ (Single Molecule Real Time) technology (Pacific Biosciences), true single molecule sequencing (e.g., HeliScope™, Helicos Biosciences), massively parallel next generation sequencing (e.g., SOLiD™, Applied Biosciences; Solexa and HiSeg™, Illumina), massively parallel semiconductor sequencing (e.g., Ion Torrent), and pyrosequencing technology (e.g., GS FLX and GS Junior Systems, Roche/454). In a preferred embodiments, sequencing may be through sequencing by synthesis technology (e.g., HiSeq™ and Solexa™, Illumina). Samples may be loaded onto a HiSeq system. The density of read clusters on Illumina flow cells can be to be optimized for cfDNA, driven in particular by the length distribution of the reads and cluster density may be optimized experimentally by sequencing various loading concentrations. The number of samples that can be loaded per cell can be defined by an analytical formula that calculates efficient utilization of each sequencing run: this is the maximum number of samples that can be run concurrently such that the desired over-amplification factor is achieved. The above concentrations result in optimal cluster generation on HiSeq2500. However, if desired cluster generation is 850-1000 K/mm² on a rapid run is not obtained the loading concentration can be varied accordingly.

### Analysis

Systems and methods of the invention are based on the insight that high-accuracy PCR enzymes are less error-prone than next-generation sequencing machines: if high-fidelity sequencing is the aim, it is therefore a good idea to create multiple copies of each individual molecule, sequence these separately and then create a consensus sequence, reflecting the sequence of the original molecule and averaging out (most) errors created during the sequencing process. A primary challenge with this method is grouping the sequenced molecules according to which original molecules they are derived from. This may be accomplished by bio-chemical labelling of the original molecules with random nucleotide sequences prior to amplification so that all sequenced molecules that share the same labelling sequences are assumed to come from the same original molecule. In preferred embodiments of the invention, sequenced molecules may be grouped without biochemical labelling; instead, statistical and bioinformatics approaches may be used to identify the progenitors of each original molecule.

These concepts can be applied to identify the columns of a BAM alignment file that are likely to contain mutated (low-frequency) alleles. The BAM format is a binary format for storing sequence data. The concept of Ensemble consistency checking, can be applied to check putative variation identified in de Bruijn graphs built from libraries, by looking for consistency in ensemble strand balance for compatible sequences.

An ensemble in accordance with embodiments of the invention is a collection of aligned read pairs. In some embodiments, an ensemble comprises a collection of aligned read pairs that share the same start and stop coordinates. In other words, for each read pair, there is a set of coordinates of reference genome coordinates that bases of the read pair are aligned to; each such set has a maximum and a minimum; an ensemble is the set of read pairs with identical maximum and identical minimum. In some embodiments, an ensemble comprises a collection of aligned read pairs that have approximately identical start and stop coordinates. Ignoring sequencing error, an individual ensemble contains the reads deriving from the PCR products of original molecules with identical, or approximately identical start/stop coordinates in the reference genome. Importantly, both strands of the original molecules should be represented as members of the ensemble, and the two source strands can be distinguished by examining whether it is the first or the second read (in an Illumina paired-end paradigm) that forms the “left” (meaning: lower reference coordinate) of an ensemble.

The over-amplification factor, discussed above can be thought of in terms of the average number of reads derived from each original molecule. If sequencing and PCR were perfect and all original molecules were unique, the number of reads per ensemble would be equal to the over-amplification factor.

Although the over-amplification factor can be determined experimentally, in preferred embodiments, it may be statistically estimated from the input BAM file. The estimation procedure can be based on the insight that most original molecules are unique, and that most ensembles should thus contain a number of reads similar to the over-amplification factor (i.e., a first approximation of the over-amplification factor can be calculated by determining the mode of a histogram that plots the number of reads per ensemble on the x axis vs the number of ensembles with that number of reads on the y axis).

To transform a set of read pair alignments into a list of ensembles (where each ensemble contains a set of read pair alignments), the ensemble definition given above can be used: all read pair alignments with identical maximum/minimum coordinate become part of the same ensemble. Importantly, this definition is based on the maximum/minimum of the complete pair alignment, and not on the maxima/minima of the 2 individual reads (that is, “inner” ends of the 2 individual read alignments can be ignored). Sequencing errors at the beginning and end of a read alignment (in aligned coordinates, corresponding to the beginning of the two individual member reads as produced by the machine) will lead to the erroneous reads forming their own ensembles. Additionally, only read pairs that satisfy a range of consistence criteria are considered based on criteria such as:
Both member reads mapped and on the same chromosome.
Opposed strands for read 1 and read 2.
Total distance between minimum and maximum of both alignments <constant (i.e., length of the supposed underlying molecule <constant; constant typically ˜330).
All quality controls flags set by the aligner (Burrows-Wheeler; Li H. and Durbin R. (2009) Fast and accurate short read alignment with Burrows-Wheeler Transform. Bioinformatics, 25:1754-60. [PMID: 19451168], incorporated herein by reference) OK for both read alignments; there is one “QC” flag and one “proper pair” flag.
Minimum mapping quality (such as >0.95).
Proportion of mismatches across the reads <constant (measured individually).
None of the members reads has soft-clipping or padding.
Genome consistency: based on the assumption that the original molecule comes from a relatively normal human genome, it is required that the read (of a read pair) that aligns to the plus strand of the reference genome be to the “left” of the other read (as measured by the minimum coordinate of their respective alignments)—and vice versa.

Which of the two strands of the original molecule ensemble members come from may be determined by examining whether the “left” read of an ensemble (as defined above) is the first or the second read of a read pair. In one preferred embodiment, an alignment algorithm where both reads of a pair have contiguous alignments is used (e.g., non-split-read alignment algorithms). In some embodiments, a split-read alignment algorithm is used (e.g., bwa mem).

Methods of the invention, including data analysis, may be conducted by a computer comprising a tangible, non-transient memory coupled to a processor. Beginning with an input BAM file, one or more of the following analysis steps may be carried out using the computer:

1. Ensemble enumeration:
All ensembles present in a BAM are identified, and their coordinates (and covariates such as length, GC content, and number of members reads) may be written into a text file (for example, clusters.txt). After outputting the file, all ensemble data can be deleted from working memory.
2. Statistical estimation of over-amplification:
A computer script (e.g., R script) that reads clusters.txt and estimates a statistical model for over-amplification can be called, taking into account covariates like GC content, ensemble length, overlap with pulldown probes. The distribution over input molecule lengths and input molecule genome coverage are also estimated.
3. Deterministic analysis:
All columns of a BAM file may be iterated through and those which are likely to contain mutated alleles are identified. Each allele in a column is a member of a cluster, and the alleles are grouped by cluster membership and by which strand of the original molecule they come from. The thresholds for identifying columns with likely mutations take into account the estimates from the statistical over-amplification model.

Probabilistic Analysis:

For candidate columns, or for all columns, a full model of PCR amplification may be applied that explicitly considers different scenarios of amplification error (at different cycles of PCR, and relative to different strands of the original molecule) and compares their likelihood with different scenarios of mutated input alleles.

Deterministic and probabilistic analysis algorithms may be column-based, i.e., they identify columns in a BAM alignment file that putatively contain mutated alleles.

Globally valid ensemble IDs for each individual read allele may be assigned or ensemble IDs may be constructed “on-the-fly”. The “on the fly” generated ensemble IDs can only be assumed to be unique/valid within each BAM alignment column, and they have no defined meaning with respect to “global” ensemble lists. The functions can be callback-based: that is, they get a function reference as an argument, which they will call for each column in the BAM alignment.

They may also be multi-threaded (i.e., parallelized based on any suitable parallelization framework (e.g., using openMP)), processing different stretches of the BAM file in parallel. The callback-functions preferably do not attempt to access global variables, or use protected memory access. The callback functions can also receive the thread number they are called from as an argument, which can also be used in constructions that avoid concurrent memory access (example: construct a vector with 16 elements if there are 16 threads, and each thread only accesses its corresponding element).

Columns, as seen by the callback functions, may be modelled as vectors of allele context objects where each of the allele context objects represents one read in the alignment. Typically one read is equivalent to one base, but if there is a local insertion, the allele context object can also contain more than one base. Apart from the raw read base(s), an allele context object can also contain the associated base qualities, further information on the alignment (mapping quality, position in read, first or second read, etc.), and, importantly, an ensemble ID that specifies which ensemble the read belongs to (this ID is locally or globally unique, see above).

The basic underlying algorithm for constructing these column vectors is an intensive process and can work as follows:
For a reference genome region, empty allele context objects may be constructed for each reference genome position.
For the same region, all read alignments from the BAM file can be obtained.
All alignments may then be deflated; that is, which bases in the raw read are aligned to which reference genome positions can be computed (this information is encoded in a CIGAR string or a sequence of base lengths and an associated operation).
For each base in the read, the base (and potentially further information on it) can be attached to its corresponding reference genome position vector.

The deterministic algorithm may be applied on a per-column basis and use the BAM access functions described above. The aim of the deterministic algorithm is to identify columns that putatively contain an admixture of mutated alleles. The analysis algorithm may function as follows:
Low-frequency alleles may be found in the column, which are treated as potential admixture alleles.
For each potential admixture allele:
   All alleles in the column can be grouped according to their ensemble ID.
   For each ensemble:
      the support can be computed (i.e., the variant allele frequency) for the variant allele from reads in the ensemble, separately for the plus and the minus strand of the underlying molecules (i.e., strands where the alignment of the first read of the read pair start at the left-hand side of the ensemble).
      each ensemble represents a number of original molecules, which can be estimated by dividing the total reads in the ensemble by the average calculated over-amplification factor.
      Ensemble may be classified as “putatively variant-allele containing” if
         the frequency of the putative variant allele is >=(1/‘estimate for the number of underlying molecules’)×factor, where factor may be a coefficient such as 0.9.
         This criterion can be required to be met separately for the reads originating from the plus and minus strands of the original molecules.
         In addition, observation a minimum number of reads from both individual strands may be required. In preferred embodiments, at least 2 reads for each original strand may be required.
   A column can be classified as “putatively variant-allele containing” if there is at least one ensemble that is classified as “putatively variant-allele containing”.

The probabilistic algorithm can also be applied on a per-column basis. The aim of the algorithm is to compute the strength of evidence for the hypothesis that a column contains an admixture of mutated alleles. As such, it is preferably employed as a second step after identifying candidates with the deterministic algorithm (the probabilistic algorithm can be computationally expensive, so minimizing its application through initial screening can be desirable). The algorithm, however, can also be used alone, without the deterministic algorithm above. In certain embodiments, the probabilistic algorithm is concerned with determining the likelihood that a candidate variant is a true variant. The probabilistic algorithm may use any known likelihood maximization model, such as, e.g., expectation-maximization, maximum likelihood, quasi-maximum likelihood, maximum-likelihood estimation, M-estimator, generalized method of moments, maximum a posteriori, method of moments, method of support, minimum distance estimation, restricted maximum likelihood estimation, or Bayesian methods.

In preferred embodiments, the probabilistic algorithm may be applied as follows:
For each column, the stored column data can be restored.
Putative mutations can be identified (e.g., by finding low-frequency variant alleles, as in the deterministic analysis above).
Alleles may be grouped in the column by ensemble ID.
For each putative mutation:
   The likelihood of the observed data can be computed under the hypothesis that the frequency of the variant allele is 0 (H0) vs a range of hypotheses that specify nonzero variant allele frequencies, with the specific frequencies generated around the empirical (column-wide) observed variant frequency.
   The likelihood calculation can proceed on a per-ensemble basis, where it is assumed that ensembles are independent (conditional on a specified variant allele frequency parameter). To obtain the likelihood of the observed data under a hypothesis, the per-ensemble likelihoods can be multiplied under that hypothesis.
   The non-zero variant frequency hypothesis with the highest likelihood can then be selected (akin to maximizing the likelihood for variant frequency, but with reduced computational strain) and a likelihood-ratio test can be carried out against H0 to obtain a p-value.
For each column, the putative mutation with the lowest p-value can be reported.

The likelihood of an ensemble can be computed under the hypothesis that there is a variant allele with a specified frequency (which can be 0). As the likelihood of a column is computed as the product of per-ensemble likelihoods, the approach described here may form the core of the probabilistic analysis approach. Each ensemble originates from an unknown number of underlying molecules. Observed variant alleles in the ensemble can either originate from truly mutated underlying molecules, or they can appear due to sequencing and PCR error. Truly mutated alleles should be equally represented on reads originating from the plus and minus strands of the original molecules. PCR errors have a different structure depending on the PCR cycle that they occurred in (earlier errors affect more molecules). Sequencing error is assumed to happen randomly (i.e., there is no particular structure about them).

The statistical model for distinguishing these scenarios can be based on the assumption of perfect PCR efficiency, i.e., each round of PCR leads to a doubling of the original molecules. This means that each strand of the original molecule and its derived molecules can be represented as a bifurcating tree (i.e., two bifurcated trees for each original double-stranded molecule)—nodes representing molecules and edges the process of PCR amplification. The number of levels in the trees is equal to the number of PCR rounds+1 (with the original molecule node representing level 1). An error model can be assumed that acts on the edges of the tree, i.e. each edge either represents accurate amplification, or an error. If an error occurs, it affects all nodes below the affected edge. Errors flip the allelic state of the molecule between ‘non-variant’ and ‘variant. The tips of the tree represent the molecules after PCR amplification, i.e. the population of molecules that go into the sequencing machine. As each ensemble originates from an unknown number of original molecules, each ensemble can be associated with an unknown number of bifurcating trees.

Based on an unknown number of unknown molecules, an unknown number of errors, etc., there is an infinite number of possible scenarios. To restrict the space of considered alternatives, the following pragmatic assumptions can be made: the number of underlying original molecules per ensemble is between 1 and 8; the number of PCR cycles is 4; and the maximum errors during the amplification of one original molecule is 2. These assumptions may be used to limit the number of scenarios considered as follows:
Between x=1-8 original molecules (i.e. 2-16 bifurcating trees)
   Of which y=0<=x can “truly” carry the variant allele
      All of which go through 4 cycles of PCR
         With z=0-2 errors along all trees for the complete ensemble
         Each of the z errors falls on one defined edge.

For each ensemble, the total likelihood may be split into 2 components: the total number of reads present in the ensemble, and the variant allele frequencies in the reads that originate from the plus and minus strands of the original molecule, respectively. This factorization can be used to reach another simplification.

It can be assumed that the effect of PCR errors on the frequency of the variant allele across the tips nodes of the tree (i.e., the sequenced molecules) can be modeled, separately for original plus and minus strands (“error_strand”), by specifying on which level of the tree the error occurred (“error_level”), and whether it affects an ancestor of a molecule carrying the variant allele (“error_variant”). A formal definition of “scenario” can be given as a combination of x, y and z values (within the boundaries specified above) plus (error_strand, error_level, error_variant) sets for each of the z errors. For a full probabilistic evaluation the likelihood of the data under all scenarios may be computed.

Each scenario has associated variant-allele frequencies at the tips level of the contained trees, separately for plus- and minus-strand deriving molecules, conditional on x, y and the error sets. A computer may be used to process this information as follows:
F_mutatedAllele_plus can be defined as the frequency of the mutated allele across the ensemble members that originate from the plus strand of the original molecules (under the assumption that the considered scenario is true) and F_mutatedAllele_minus as the frequency of the mutated allele across the ensemble members that originate from the minus strand of the original molecules.
F_mutatedAllele_plus:=F_mutatedAllele_minus:=y/x may then be initialized.
For each of the z errors defined as (error_strand, error_level, error_variant):
   levels_downstream_affected:=roundsPCR−error_level; (1-based level indexing, i.e. an error in the first round of PCR has level 1).

$$oneMutation\_effect := \frac{{2}^{levels \_ downstream \_ affected}}{{2}^{roundsPCR - 1}} \times \frac{1}{x}$$

If error_strand=“+”:
   If error_variant=“non variant”:
      F_mutatedAllele_plus=F_mutatedAllele_plus+oneMutation_effect
   If error_variant=“variant”:
      F_mutatedAllele_plus=F_mutatedAllele_plus−oneMutation_effect
If error_strand=“−”:
   If error_variant=“non variant”:
      F_mutatedAllele_minus=F_mutatedAllele_minus+oneMutation_effect
   If error_variant=“variant”:
      F_mutatedAllele_minus=F_mutatedAllele_minus−oneMutation_effect
F_mutatedAllele_plus and F_mutatedAllele_minus can be restricted to boundaries of 0 and 1.

In various embodiments, for each of the z errors, the program may optionally only specify a. whether it affects an ancestor of a molecule carrying the variant allele (“error_variant”); b. whether it affects an ancestor of a plus- or minus-strand original molecule (“error_strand”); and/or c. the tree level of the error (“error_level”). In certain embodiments, the program may specify a. which of the 1 . . . x molecules (+ ancestors) the error affected; b. whether it affected the ancestors of the original plus or minus strand; and/or c. precisely on which edge of the corresponding tree the error occurred.

To compute a likelihood of the data under each considered scenario, a prior scenario likelihood can be obtained and multiplied by the likelihood of the data under the scenario. Each scenario can be given a prior probability as follows: X can have a probability distribution from the output of the statistical estimation of over-amplification computer script, taking into account the original molecules genome coverage, conditional on the length of the ensemble (e.g., longer ensembles have a higher chance of originating just from one original molecule). y can have a (Poisson) probability distribution, parameterized by the frequency of the assumed variant allele. z, the total number of errors, may have a (Poisson) probability distribution (from the experimentally estimated error frequency of the PCR enzyme scaled by the number of edges), and assume that each edge is equally likely to be hit by an error (i.e., ancestors of variant-allele-carrying and non-variant original molecules are hit with probabilities proportional to the number of these molecules in the scenario (variables x and y). Only tracked considerations in this scenario are whether the error hits a variant/non-variant-molecule ancestor tree, whether it hits a plus/minus strand tree, and which level it hits (as described above).

The data for an ensemble can be given a likelihood based on the scenario. It can be noted that the ensemble data consist of alleles with associated quality values (usually a FASTQ base quality), and that each allele is either identical to the variant allele or not (‘non-variant’). Furthermore, for each considered scenario, the frequencies for variant alleles at the tips level of the trees can represent ancestors of the plus and minus strands of the original variant and non-variant molecules.

Using these frequencies, the observed ensemble data as may be modelled as Bernoulli distribution (separately for plus and minus strand ancestors), integrating over individual allele base qualities.

The class likelihoodTree <int roundsPCR, int maximumUnderlyingMolecules, int maximumErrors> represents the collection of all scenarios for a given variant allele frequency. That is, to carry out a full analysis, comparing H0 (variant allele frequency=0) against multiple hypotheses, multiple likelihoodTree objects may be required. The basic scenario parameters, such as rounds of PCR, maximum underlying molecules, and maximum number of errors per ensemble, may be represented as template arguments, enabling efficient compiler optimization.

The class likelihoodBranch <int roundsPCR, int maximumErrors > represents individual scenarios, consisting of the following information:
The total number of underlying molecules
How many of these underlying molecules carry the variant allele
How many errors there are:
   On each level of the trees representing the ancestors of the plus-strand non-variant original molecules
   On each level of the trees representing the ancestors of the plus-strand variant original molecules
   On each level of the trees representing the ancestors of the minus-strand non-variant original molecules
   On each level of the trees representing the ancestors of the minus-strand variant original molecules

The method likelihoodBranch::likelihood_data(..) can compute the likelihood of one ensemble under the scenario represented by the likelihoodBranch object.

A likelihoodTree object needs to be populated with all consistent likelihoodBranch objects. The function likelihoodTree::computeErrorConfigurations(..) computes all consistent scenarios, which are then (in the constructor likelihoodTree) transformed into likelihoodBranch objects. The prior probability of each scenario may also be computed in the likelihoodTree constructor.

An R component can help determine the probability distribution over the number of underlying molecules for an observed ensemble of a specified length, GC content etc. and with a specific number of reads. In order to answer this question estimates for the following quantities can be derived:
Conditional on an ensemble length, infer the a priori probability distribution over the number of underlying molecules. This distribution is influenced by the total genome coverage of the underlying molecules and their length distribution (which therefore needs to be estimated).

Conditional on assuming that there is a specific number of underlying molecules, infer the probability distribution over reads generated by these underlying molecules.

This distribution is influenced by the properties of the over-amplification process, which is assumed to act independently on the original molecules and which is assumed to follow a Poisson distribution.

For each individual original molecule, the mean of the Poisson can be parameterized by (the exponential of) a linear function with an intercept (Mu) and coefficients for
The length of the ensemble (Length).
The deviation of the ensemble's GC proportion from 0.5 (GCm50).
The extent to which the ensemble has less than 90 bases overlap with the nearest pull-down probe (PulldownLess90), if pull-down capture enrichment has been applied. A similar metric can be constructed for other methods of enrichment such as those discussed earlier.

Quantity estimations described above may be performed using a probability distribution of the number of underlying molecules per ensemble.

This probability distribution may form a matrix with ensembles in rows and possible numbers of underlying molecules as columns where each row sums up to 1. This probability distribution can be initialized by considering the histogram over reads per ensemble: in the application of cfDNA sequencing from blood plasma most molecules may be considered to be unique (as indicated by in silico simulations using the molecule length distribution from sequencing data obtained from whole genome PCR-free cell free DNA sequencing), accordingly, the majority of ensembles can have a number of reads equivalent to their achieved over-amplification factors. To take into account the influence covariates, the ensemble data can be stratified by covariate value (in multi-dimensional quantiles), and then the procedure may be carried out for each quantile separately. This provides a first-guess over-amplification factor for each ensemble. The matrix can be populated by assuming that observed read count follows a Poisson distribution, with mean equal to number_underlying_molecules×over-amplification_factor_of_ensemble. The matrix may be filled in a row-wise fashion with the attained likelihoods, and normalize by row. This provides a first approximation of the probability distribution over underlying molecules for each ensemble.

The distribution may be refined by employing an expectation-maximization (EM) like procedure to refine the probability matrix. Some simplifying assumptions of independence can be made in this process.

For the EM algorithm, the assumption that observed read count follows a Poisson with mean number_underlying_molecules×over-amplification_factor_of_ensemble can be kept, but over-amplification_factor_of_ensemble can be replaced by exp(over-amplification(Mu, Length, GCm50, PulldownLess90)) where over-amplification(Mu, Length, GCm50, PulldownLess90) is a linear predictor of over-amplification factor for individual molecules. over-amplification(Mu, Length, GCm50, PulldownLess90) may be computed individually for each ensemble, taking into account the global coefficients as well as the ensemble's individual values for GC content, pulldown overlap etc.

For the EM part, prior probabilities can be introduced on the columns of the matrix, conditional on ensemble length (i.e., each ensemble has its own column-wise priors). These prior probabilities depend on the starting rate of original molecules at each position of the genome (coverage) and the molecule length distribution, quantities which may also be estimated—and are assumed independent of over-amplification covariates conditional on a fixed per-ensemble underlying molecule number probability distribution. The estimation procedure is described in more detail below.

The EM-like algorithm may be structured as follows:
1. Initialize E=clusterData_P_underlying
2. (M step) Keeping E fixed, estimate underlying molecule genome coverage, length distribution and prior distribution over the number of underlying molecules, conditional on ensemble size.
3. (M step) Keeping E fixed, estimate Mu, Length, GCm50, PulldownLess90.
4. (E step) Keeping Mu, Length, GCm50, PulldownLess90 and the underlying molecules prior distribution fixed, estimate E from the observed data (number of reads per ensemble).
5. Measure the likelihood of the observed data under E and all parameter estimates; go to Step 2 if sufficient improvement, abort if not sufficient improvement.

Estimating genome coverage and length distribution of underlying molecules and prior probabilities on number of underlying molecules per ensemble may be accomplished using a populated matrix that specifies a probability distribution over numbers of underlying molecules for each ensemble. The starting rate of underlying molecules per position can be estimated, then length distribution, and then the prior distribution conditional on ensemble length.

### Starting Rate/Coverage Estimation

First positions can be identified at which to measure coverage. In certain embodiments, only coverage at positions that exhibit sufficient overlap with pull-down probes may be measured (or more precisely: the overlap of hypothetical cfDNA molecules starting at these positions with the pulldown probes needs to be sufficient). If too many positions are identified, the ensemble data can be down-sampled to include only ensembles starting at a subset of the positions (that is: all ensembles which do not start at one of these positions are removed). This sub-sampling can be carried out once prior to entering the EM parts of the algorithm and affects all steps of the estimation procedure, including estimation of Mu, Length, GCm50, PulldownLess90. An estimate for the starting rate of molecules can be derived by identifying all ensembles that start at one of the selected positions and summing over their expected number of underlying molecules. This number can then be divided by the number of considered positions. If required, a coverage can later be obtained by multiplying by average molecule length.

### Length Distribution Estimation

For each ensemble, the expected value of underlying molecules can be inferred. A weighted average of ensemble lengths can then be calculated (weighted by the underlying molecules estimate for each ensemble). Missing values (e.g. caused by the subsampling during the “Coverage” part) may be interpolated.

### Estimation of Prior Distribution of the Number of Underlying Molecules Per Ensemble at a Specified Length

Starting rate per position and the distribution of lengths can enable calculation of the prior probability on the number of original molecules underlying an ensemble of specified length. First one may iterate over the number x of possible starting molecules at a position (which is independent of length), and then calculate the probability that y<=x=1, 2, 3 . . . of these molecules are equal to the specified length of our ensemble (from the length distribution). The probability distribution can then be normalized over possible (x, y) values and marginalize for y.

According to certain embodiments, systems and methods of the invention may include a simulator. The simulator function may take an input which specifies parameters such as coverage, mutated allele admixtures, and the selected bins. The two most important parameters are coverage of the “raw cfDNA” product pre-PCR and envisaged sequencing data coverage. (measured over our regions of interest, see below). Coverage of the “raw cfDNA” product pre-PCR comprises molecules from the mutated subclones (see below) as well as non-mutated molecules. The spread between the two parameters may be used to determine the over-amplification factor. In certain embodiments, the simulation process may be characterized by the following properties:
Simulated genomic regions may be limited to the regions captured by the pull-down panel.
A number of mutations are spread over the specified region. Each mutation has an associated admixture frequency (the frequency of which it is present in our simulated cfDNA). Each admixture frequency may be treated as a separate subclone, and therefore all mutations belonging to one bin are simulated together (i.e., they would form haplotypes, if they were close enough to each other).
A molecule pool can be created representing a total cfDNA product (i.e., including mutated and non-mutated fragments). The pool may be populated by separately simulating molecules originating from the non-mutated reference genome, and from the specified subclones (i.e.: from the specified admixture frequencies). If a molecule originates from a non-reference subclone, it (if it overlaps) carries the mutations associated with its source subclone/admixture frequency. Total coverage for each part of the simulation procedure can be determined by spreading the total desired coverage of the pre-PCR product over the different subclones (with specified admixture proportions) and the non-mutated reference genome (receiving the remaining, non-admixed proportion).
Below are two examples for how coverage is spread between subclones and the non-mutated reference genome:
   If a desired total coverage of 1,000× of original molecules is specified, and if there is one subclone/admixture bin with 10% frequency the following coverages are obtained: 900× for the “non-mutated” and 100× for the “10% admixture” subclone.
   If an additional admixture bin with 1% frequency is added the following molecule bins are found: 890× non-mutated, 100×10% mutated, 10×1% mutated.
Control sequences with pre-defined sequences can also be added to the molecule pool (as a first step after creating the pool). Each control sequence can be represented by multiple identical molecules, and the number of identical molecules per control sequence may be drawn from a Poisson distribution (the mean of which can be user specified and can be different for different control sequences).
After populating the molecule pool, the ligation of P5 and P7 adaptors and PCR amplification may be simulated (separately for plus and minus strands and preserving the orientation of the ligated P5/P7 molecules). The simulation can be carried out on the pool, i.e., the number of molecules in the pool grows with each simulated round. The PCR process simulation can comprise the simulation and sequencing errors and imperfect amplification. The probability of imperfect amplification can be calculated individually for each molecule in the pool and depends on the GC content of the molecule. The number of PCR cycles may be calculated from the desired sequencing read coverage and the specified sequencing efficiency. The required coverage in the molecular pool post-PCR can be calculated by multiplying the desired sequencing coverage by 1/the specified sequencing efficiency. Then, taking into account average amplification efficiency (of the molecules in the pool pre-PCR), one can calculate how many cycles of PCR are required to bring the coverage in the pool from the pre-PCR level to the desired post-PCR level.
   To give an example for this calculation, if the total envisaged coverage is 160,000, and the total original molecules are estimated at 20,000 (i.e. coverage of the original molecules of 20,000× over the regions of interest), and PCR efficiency is 100%, and specified sequencing efficiency is 0.5: a coverage of 320,000× in the post-PCR pool is needed, and this requires 4 cycles of PCR.
Finally, one can sample from the post-PCR molecules in the pool (with a sequencing efficiency rate), and generate paired-end sequencing reads for each selected molecule. P5/P7 ligation orientation determines which end of the molecule generates the 1^{st} read. The generation of sequencing reads can include the simulation of sequencing errors.

The simulator can keep track of many of the important events, e.g. the location and timing (which PCR round) of PCR errors. These data can be stored as text files in a simulation output directory.

After the simulation of sequencing reads has finished, the simulated reads can be mapped to the reference genome. After mapping has finished the data can be analyzed and used to produce an analysis of how many of the simulated mutations were called and how many false-positives there were. This output may be sent to an input/output device such as a printer or display.

In preferred embodiments, analysis of sequencing data may begin with a BAM file as input data with the output being one or more text files.

### Controls

In certain aspects, systems and methods of the invention relate to estimating the impact of sequencing error, and non-uniform coverage, on variant allele frequency estimates using somatic alterations in the sample. To do this, a somatic variant that has N consecutive bases differing from the germline genome (N>1) can be identified and represented by the vector V={a(1), a(2), . . . , a(n−1), a(n)}, where the elements a(i) represent the differing base at location i in the variant. Such variants can be generated by somatic alterations: translocation, inversion, insertion, deletion, amplification.

For each base a(i) in the variant count the total number of alleles supporting that base, this generates n estimates of the frequency of V, f(V). All the observed frequencies f(a(i)) should equal f(V), but due to variation in coverage and sequencing error this may not be the case. Known statistical methods can then be used to quantify the dispersion in frequency estimates that arise during sequencing. This can then be used to correct frequency estimates. One example would be to use the sample mean and variance to estimate a confidence interval using an appropriate sampling distribution.

The ratio of alleles at heterozygous sites should be 1/2 in diploid organisms. There exist large databases of SNPs segregating in human populations. For a given individual, these sites can be interrogated and heterozygous sites identified as loci with two alleles with roughly equal allele frequencies. An empirical distribution of allele frequencies can then be constructed from the observed frequency of the second allele at the heterozygous sites. If the number of heterozygous sites is large enough, frequency estimates can be constructed per allele combination (A>C, A>G, . . . , T>G). The distribution can then be used to correct frequency estimates at the somatic variant sites in sample data.

A known input amount of DNA, that has distinct sequence from the patient may be added to the sample in certain embodiments. These are positive controls for variant alleles in the sample. To generate an identifiable spike in sequences that are unlikely to be observed in the human population can be generated. This may be done by 1) choosing regions that have low reported diversity in population sequencing databases, 2) introducing changes to the sequence that do not reflect natural mutation processes (e.g. the sequence (same)n,{change, same, change, same, change},(same)n). The control sequence can be further distinguished because the length of the spike-ins (120 bases) is known and so are the location of the introduced changes.

It is known that hybridization capture can be impacted by the number of mismatches between the capture probe and the target DNA. In certain embodiments then, 4 mutations are introduced into each control. Spike-ins can also be constructed so that the impact of 1) GC-content and 2) probe-target overlap can be observed by 1) choosing sequence with differing GC-percentages from the known GC-content distribution across the targeted regions and 2) varying the percent overlap of the 120 base long control DNA with its corresponding pull down probe.

The spike ins can be added to the blood collection vacutainer before blood draw so that a) samples can be identified from their sequencing allowing the identification of sample mix-up in the sequencing, b) so that contamination from apoptosis of nucleated white blood cells can be estimated, and c) so that false negatives can be detected.

Cell-free circulating DNA from human blood plasma (pDNA) contains, besides a majority proportion of molecules derived from a person's normal (typically healthy) genome, fragments of tumor DNA in cancer patients and fragments of fetal DNA in pregnant women. Surveying that admixed portion of either tumor or fetal DNA is intrinsically challenging, for the admixture proportion of the cancer-/fetus-derived molecules can be as low as 1 in 5000 molecules.

Any given unprocessed blood sample, typically but not always stored in an EDTA tube or different type of blood collection vessel, will contain a certain fraction of cell-free DNA as well as white and red blood cells (WBCs and RBCs). After a period of time (and influenced by environmental factors such as temperature), the contained WBCs will undergo cell death and start releasing the contained DNA fragments into the circulation. Due to the process, any tumor- or fetus-derived cell-free DNA contained in the blood sample will be further diluted, rendering their detection and characterization even more challenging.

Technical solutions (e.g. Streck tubes) exist to prevent the contained WBCs from rupturing and releasing their DNA, but these solutions are not perfect and the problem of dilution remains, in particular if the blood is stored for longer periods of time or when the blood sample is transported.

For any diagnostic method based on surveying the existence or characteristics of tumor- or fetus-derived DNA, it is thus desirable to measure and control for potential contamination.

In certain embodiments of the invention, synthesized perturbed DNA may be spiked into collection vessels to track contamination. A stretch or a region in the human genome can be determined that is a) homozygous in the vast majority, i.e., has a known and/or ascertainable frequency threshold of the human population (or homozygous in the vast majority of the desired target population) and b) high in genomic complexity, i.e., establishing the genomic origin for molecules derived from that region is, using standard algorithmic methods for read alignment, unambiguous and unchallenging. Typically, that stretch would vary in length between 50 and 150 bases, but the method described here can utilize both longer and shorter regions. The sequence of the stretch or region may then be perturbed by either substituting a number of nucleotides with different nucleotides or introducing or deleting a number of nucleotides. Typically this step would include the substitution of one or two nucleotides located centrally in the sequence with different nucleotides. Next it may be verified that the perturbed sequence is not present in the normal human population. A variety of standard methods exist to achieve this, for example genome alignment or comparison to a de Bruijn graph generated from population sequencing data, such as the 1000 Genomes Project. If this verification fails, Steps 2 or 1 can be repeated.

The perturbed sequence may then be synthesized to produce (approximately or exactly) n copies of the so-perturbed sequence using DNA synthesis methods. Typically the number n is chosen so that, when the n molecules are introduced into the patient sample, in this case blood, the ratio between n and the expected number of copies of the human genome in the volume of blood drawn resembles the expected/desired minimum ratio between tumor-/fetus-derived fragments and normal genomic fragments. (E.g. if one expects 1 in 1000 circulating fragments to be of tumor origin, and if one ml of blood typically contains about 1000 copies of the human genome, and one draws 5 ml of blood, n=5 per tube would be a sensible choice).

The synthesized copies of the perturbed sequence can be present in a collection vessel prior to collection or may be added to a sample after collection. The synthesized perturbed DNA contacts the sample at time X. During analysis of the sample, the cell-free circulating DNA may be extracted by centrifugation and a DNA library can be prepared from the extracted DNA. The observed frequency of the perturbed sequence (f_{P}) and of the frequency of the unperturbed sequence (fₙ), may be measured using the technology that will be used in downstream interpretation of the sample (e.g., a digital PCR-based approach or a sequencing-based approach, either utilizing a whole-genome sequencing method or a targeted sequencing approach)

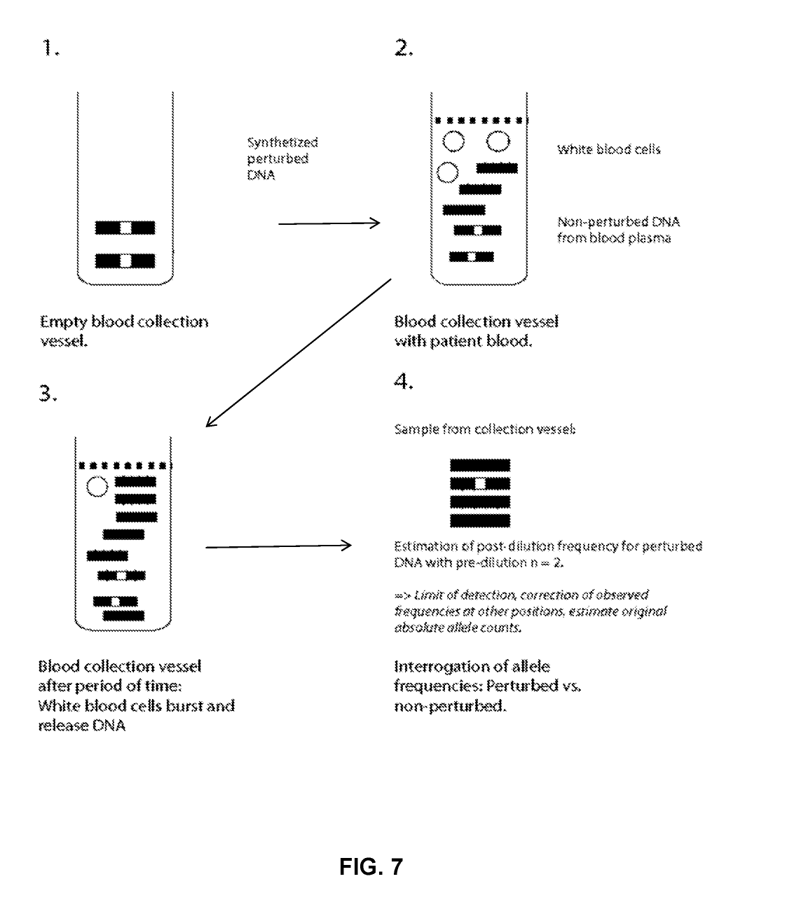

The observed frequencies may be analyzed as follows: f_{P}/(f_{P+}fₙ) is an estimator for the post-dilution frequency of tumor- or fetus-derived alleles originally (i.e. before dilution due to rupturing WBCs started) present at n copies in the sample. Depending on the properties of employed downstream interpretation technologies, if f_{P}/(f_{P+}fₙ) is 0 or below a specified threshold, the sample should be rejected or not be interpreted. Multiplying the observed number frequency of an assumed tumor- or fetus-derived allele in post-dilution data by ([(f_{P+}fₙ/f_{P})]×n will give an estimate of the pre-dilution absolute count of that allele. Tumor allele counts and their development over time have been demonstrated to be important indicators of disease status and progression. The above process using pre-seeded collection vessels is exemplified in FIG. 7.

The above procedure may be used for different genomic loci and different values of n to confer additional advantages such as controlling for GC content bias and enabling the (more accurate) estimation of the total amount of dilution (measured in dilution-derived molecule fragments) and hence the pre-dilution number of DNA fragments in the blood sample.

A computer, as referenced herein, generally includes a processor coupled to a memory and an input-output (I/O) mechanism via a bus. Memory can include RAM or ROM and preferably includes at least one tangible, non-transitory medium storing instructions executable to cause the system to perform functions described herein. As one skilled in the art would recognize as necessary or best-suited for performance of the methods of the invention, systems of the invention include one or more processors (e.g., a central processing unit (CPU), a graphics processing unit (GPU), etc.), computer-readable storage devices (e.g., main memory, static memory, etc.), or combinations thereof which communicate with each other via a bus.

A processor may be any suitable processor known in the art, such as the processor sold under the trademark XEON E7 by Intel (Santa Clara, Calif.) or the processor sold under the trademark OPTERON 6200 by AMD (Sunnyvale, Calif.).

Input/output devices according to the invention may include a video display unit (e.g., a liquid crystal display (LCD) or a cathode ray tube (CRT) monitor), an alphanumeric input device (e.g., a keyboard), a cursor control device (e.g., a mouse or trackpad), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

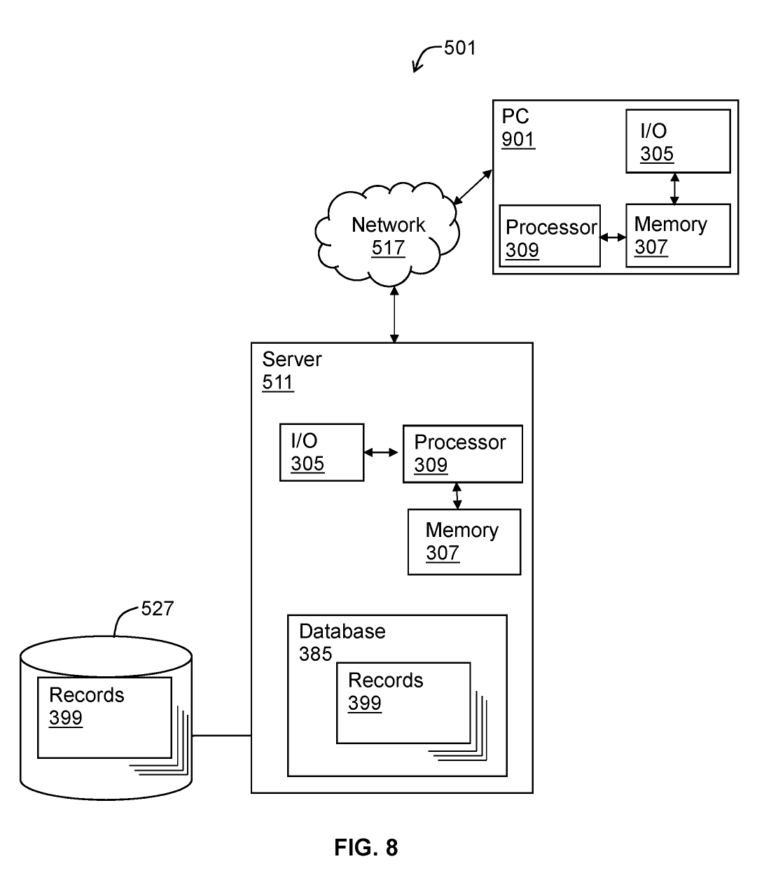

An exemplary system 501 of the invention is depicted in FIG. 8. A computer 901 comprising an input/output device 305 and a tangible, non-transient memory 307 coupled to a processor 309. In certain embodiments, the computer 901 may be in communication with a server 511 through a network 517. The server 511 may also comprise an I/O device 305 and a memory 307 coupled to a processor 309. The server may store one or more databases 385 capable of storing records 399 useful in methods of the invention as described above.

Aspects of the invention include algorithms and implementation protocols, as described herein. The SENTRYSEQ technology is based on the insight that high-accuracy PCR enzymes are less error-prone than next-generation sequencing machines: if high-fidelity sequencing is the aim, it is therefore a good idea to create multiple copies of each individual molecule, sequence these separately and then create a consensus sequence, reflecting the sequence of the original molecule and averaging out (most) errors created during the sequencing process.

Aspects of the subject methods involve identifying the columns of a BAM alignment file that are likely to contain mutated (low-frequency) alleles. The concept of ensemble consistency checking can be applied to check putative variation identified in de Bruijn graphs built from SENTRYSEQ libraries by looking for consistency in ensemble strand balance for compatible sequences.

### Ensembles

An ensemble is a collection of aligned read pairs that share the same start and stop coordinates (precise definition: for each read pair, there is a set of coordinates of reference genome coordinates that bases of the read pair are aligned to; each such set has a maximum and a minimum; an ensemble is the set of read pairs with identical maximum and identical minimum).

Ignoring sequencing error, an individual ensemble contains the reads deriving from the PCR products of original molecules with identical start/stop coordinates in the reference genome. Importantly, both strands of the original molecules should be represented as members of the ensemble, and the two source strands can be distinguished by examining whether it is the first or the second read (in an Illumina paired-end paradigm) that forms the “left” (meaning: lower reference coordinate) of an ensemble.

### Over-Amplification Factor

The over-amplification factor is the average number of reads derived from each original molecule; if sequencing and PCR were perfect and all original molecules were unique, the number of reads per ensemble would be equal to the over-amplification factor.

Although the over-amplification factor can be measured experimentally, in the current paradigm it is statistically estimated from the input BAM file. The estimation procedure is based on the insight that most original molecules are unique, and that most ensembles should thus contain a number of reads similar to the over-amplification factor (i.e., a first approximation of the over-amplification factor can be calculated by determining the mode of a histogram that plots the number of reads per ensemble on the x axis vs the number of ensembles with that number of reads on the y axis).

### Valid Ensembles

Real sequencing data contains sequencing errors, and not all reads can be mapped perfectly. To transform a set of read pair alignments into a list of ensembles (where each ensemble contains a set of read pair alignments), the definition given above is used: all read pair alignments with identical maximum/minimum coordinate become part of the same ensemble. Importantly, this definition is based on the maximum/minimum of the complete pair alignment, and not on the maxima/minima of the 2 individual reads (that is, “inner” ends of the 2 individual read alignments are ignored).

Sequencing errors at the beginning and end of a read alignment (in aligned coordinates, corresponding to the beginning of the two individual member reads as produced by the machine) will lead to the erroneous reads forming their own ensembles. In addition, only read pairs that satisfy a range of consistency criteria are considered. These can include:
Both member reads mapped and on the same chromosome.
Opposed strands for read 1 and read 2.
Total distance between minimum and maximum of both alignments <constant (i.e., length of the supposed underlying molecule <constant; constant typically ˜330).
All quality controls flags set by the aligner (BWA) OK for both read alignments; there is one “QC” flag and one “proper pair” flag.
Minimum mapping quality (currently >0.95).
Proportion of mismatches across the reads <constant (measured individually; currently constant=2, i.e. the flag is not active).
None of the members reads has soft-clipping or padding (currently no active).
Genome consistency: based on the assumption that the original molecule comes from a relatively normal human genome, the read (of a read pair) is required to align to the plus strand of the reference genome be to the “left” of the other read (as measured by the minimum coordinate of their respective alignments)—and vice versa.
   Distinguishing Between Ensemble Members from the Plus and the Minus Strand

Which of the two strands of the original molecule ensemble members come from can be distinguished by examining whether the “left” read of an ensemble (as defined above) is the first or the second read of a read pair.

### Important Technical Considerations

The criteria as defined above require that both reads of a pair have contiguous alignments; BAMs produced by alignment algorithms supporting split-read alignment, like BWA-mem, are problematic.

### Overview of the Analysis Process

Provided with an input BAM, SENTRYSEQ carries out the following steps:

1. Ensemble enumeration
Find all ensembles present in a BAM, and write their coordinates (and covariates like length, GC content, number of members reads . . . ) into a text file clusters.txt.
After outputting the file, all ensemble data are deleted from working memory.
Main function: clusterGenerator::enumerateClustersInBAM(..) in clusterGenerator/clusterGenerator.cpp.
   2. Statistical estimation of overamplification
Call an R script that reads clusters.txt and estimates a statistical model for overamplification, taking into account covariates like GC content, ensemble length, overlap with pulldown probes. The distribution over input molecule lengths and input molecule genome coverage are also estimated.
Main file: R/analyeSENTRY.R.
   3. Deterministic analysis:
Iterate through all columns of a BAM file and identify those which are likely to contain mutated alleles. Each allele in a column is member of a cluster, and the alleles are grouped by cluster membership and by which strand of the original molecule they come from. The thresholds for identifying columns with likely mutations take into account the estimates from the statistical overamplification model.
Main function: deterministicAnalysis::kickOff( ) in analysis/deterministic/deterministicAnalysis.cpp.
   4. Probabilistic analysis (not always activated)
For candidate columns, or for all columns, apply a full model of amplification that explicitly considers different scenarios of amplification error (at different cycles of PCR) and compares their likelihood with different scenarios of mutated input alleles.
Main function: probabilisticAnalysis::kickOff( ) in analysis/probabilistic/probabilisticAnalysis.cpp.

Aspects of the invention include high fidelity sequencing methods and protocols that are described in further detail below. A necessary condition for the detection and accurate frequency estimation of low abundance somatic mutations in a population of molecules (mutant allele proportion <5000⁻¹) is to maintain the ratio of derived alleles N_{d} (corresponding to somatic variants) to ancestral alleles Nₐ (corresponding to the germ-line genome) and DNA from other sources N_{?} throughout the sample preparation and library preparation process.

$$f = \frac{{N}_{d}}{{N}_{a} + {N}_{?}}$$

The proportion of derived alleles f can be decreased by (a) depleting N_{d} through losses in the sequencing library construction process, or (b) increasing the denominator through contamination.

For the application of sequencing circulating tumour DNA (ctDNA) sequencing from cell free DNA (cfDNA) samples, steps must be taken to control (a) by minimizing nuclear DNA contamination released by apoptotic cells during and/or after blood draw, and to control (b) steps must be taken to minimize the loss of molecules during library preparation.

A challenge in the detection of low frequency alleles (f< [symbol] 10 [symbol] \hat{ }(³)) is that high throughput sequencing have sequencing error rates about O(1 error/1000 base). There are known covariates of Illumina sequencing error, for example, position in read, base, homopolymer length, etc. To control error rates, PCR-duplicates of original molecules are generated and then a statistical model is used to assess evidence for true variation versus error at each detected variant aggregating over identified duplicates which are referred to as Ensembles. Ensembles are constructed de novo by scanning for shared alignment and read length to identify reads arising from potential PCR-duplicates, the fact that in the original population there can be multiple identical molecules is accounted for (the number of identical original molecules is a function of cfDNA concentration and cfDNA length distribution). The average number of duplicates for each original molecule is referred to as the over-amplification factor.

Over-amplification factor is minimized by propagating uncertainty in sequence reads covering the underlying candidate variants using a statistical model and accounting for the inferred number of underlying molecules. This has the effect of reducing the required sequencing (the main cost component) compared to other methods. As such, the library preparation protocol described herein has been jointly optimized with the statistical models that are used to identify variants and their associated statistical significance.

Aspects of the invention include methods for the preparation of sequencing libraries from cell free DNA (cfDNA) for use on Illumina sequencing platforms, apart from Library Preparation, the methods can be applied to any fragmented DNA on any shotgun sequencer. For instance, this means that minority cell populations can be detected in a population of cells by fragmenting the DNA (using e.g. restriction enzymes or sonication) and then applying the same Ensemble generation strategy.

FIG. 2 shows Illumina adapter ligation products. Protocol modifications result in adapter stacking. This is done to maximize the number of sequencing compatible products (see FIG. 3 for PCR resolution of stacked adapters). FIG. 3 shows resolution of stacked adapters through primer binding competition and resulting PCR products. If the innermost primer binds before, or concurrently with, the outermost PCR primer annealing site, the result is the elimination of the outermost primer from the PCR product. Since the waiting time for innermost binding first is geometrically distributed, after 4 rounds of PCR the chances of not obtaining a product compatible with sequencing are only 1/16.

FIGS. 4-5 show an example of a cfDNA library from a lung cancer patient. About a doubling in sequencable product is observed using this approach. In FIG. 4, four peaks are observed, the first 3 relating to the average molecule length plus 2, 3, and 4 adapters. After PCR (FIG. 5), the mode shifts to the average molecule length plus 2 sequencing adapters. Two longer fragment populations are also observed.

Aspects of the invention include methods for isolating DNA samples into two population partitions using hybridization capture techniques. Hybridization capture is a method to isolate specific DNA molecules from a population based on their nucleotide sequence. To capture molecules, the double stranded DNA is melted into single stranded DNA (e.g. by increasing the temperature), then the hybrid capture probes (probes) are added, and conditions changed to encourage strand annealing. Probes are complementary to the target sequence and have a selectable marker (e.g. biotin) that enable the molecules to be isolated. To prevent hybridization between hybridization probes in a probe pool, all the probes in a pool are designed to all complement either the sense or anti-sense sequence of the target loci. Consequently, only one strand is captured per double stranded DNA molecule.

Typically, the sample DNA is PCR amplified prior to hybridization capture, which leads to both strands of the original molecule being represented in the sense and anti-sense PCR duplicate population. For the purposes of clarity consider the following toy example: x={x₊, x₋} is a double stranded molecule, a and are single stranded DNA molecules of length l, a shares complementary sequence for its first n consecutive bases with the last n consecutive bases of β, the remaining sequence is non-complementary. Consequently, α annealed to β has a forked Y-shaped structure of a double stranded DNA stem from the complementary sequence, and single stranded DNA arms from the non-complementary sequence.

Now, create a molecule where x is flanked on either end by the Y-shaped {α, β} double stranded DNA using known ligation reactions, e.g. blunt end ligation:

αx₋β

βx₊α

Then apply PCR using primers complementary to α and β, represented as α_c and β_c respectively, generating the family of PCR duplicates:

$$α {x}_{-} β {\to }^{PCR} \{ \begin{matrix}{α}_{c} {x}_{+} {β}_{c} , & α {x}_{-} β \}\end{matrix} \\ β {x}_{+} α {\to }^{PCR} \{ \begin{matrix}{β}_{c} {x}_{+} {α}_{c} , & β {x}_{+} α \}\end{matrix}$$

Applying a hybrid capture protocol with probe sequence [symbol] x [symbol] _(−) to the PCR products, would extract molecules with sequence α_c x_(+) β_c and β [symbol] x [symbol] _+α, which each are descendants of the anti-sense and sense strands respectively. Subsequent PCR generates all four single stranded molecules, so both strands are represented. However, half of the molecules are not captured using this approach. Current capture methods only use one set of either sense or anti-sense probes.

Strand specific isolation can be used to generate two identically distributed samples from the original sampled DNA. This is useful for applications that seek to detect molecules at low frequency in a heterogeneous population as a means of controlling for errors and dropout induced in subsequent manipulation of the sampled DNA. The following two-step process is proposed:

Separately manufacture two hybrid capture panels for the loci of interest; one sense, and one antisense. The order of application of sense, anti-sense is irrelevant so call the panels A and B. Then apply the panels in series to the DNA sample as follows. Affinity selection proceeds: apply the selectable probes to single stranded DNA, separating the sample into the isolate (DNA bound by probes) partition and non-isolate partition (DNA not bound by probes) using standard hybrid capture protocols.

STEP 1: Apply A to the DNA population. The target sequence will be collected in the isolate partition. Retain the non-isolate partition.

STEP 2: Apply B to the non-isolate partition. The complement of panel 1 target sequence will be collected in the isolate partition of STEP 2.

There may be some carry over contamination of probes from A, but this should be minimal if isolation methods are optimized. In an alternative implementation, the sample could be partitioned into two aliquots and A and B applied separately, thereby avoiding any cross-hybridization that results from probe carry through in the previous step.

Analyze isolates from A and B separately, then look for concordance in the results between the two experiments, which controls for artifacts that are introduced in downstream treatment of the samples. This provides the opportunity for replication between isolates A and B, and increases sensitivity by assessing A and B separately.

Aspects of the invention include methods for carrying out hybrid capture region selection procedures. Targeted high throughput sequencing is motivated by reducing the total number of sequencing reads required to assess specified loci in an individual. The reduction in required reads is a function of the quotient targeted sequence length divided by genome length, and weights determined by the distribution sequencing read depth of coverage (henceforth abbreviated as coverage) for the targeted and whole genome sequencing.

Increased coverage improves sensitivity since the number of reads containing a target allele is approximately binomially distributed with true variant proportion (1−ε)×f, where ε is the base error rate in sequencing and f is the frequency of the allele in the molecule population and coverage D. The relationship between coverage and sequencing error is complex, but assuming no systematic error, coverage can reduce false positives by enabling aggregating information across reads spanning a target locus (integrating out errors). More complicated error models are required because systematic error modes exist in sequencing, such as errors in homopolymers.

Selecting which regions of the genome to target is an important consideration in the design of targeted sequencing panels. In the context of cancer detection using genetic variant signatures, the statistical power of the targeted panel is a function of the recurrence of variants within the patient population across those loci. An additional consideration in hybrid capture design, is the specificity of each hybridization probe and the uniformity of sensitivity across all the probes, both drive the amount of sequencing reads required to detect variants at a desired limit of detection.

Here a process is described to select the combination of loci up to a total sequence length L which optimizes for the greatest combined recurrence load in cancer patients (combining both driver and passenger genetic variants), accounting for determinants such as sequence uniqueness and GC content that affect hybrid capture performance. Furthermore, synthetic DNA spike-ins are designed, which match cfDNA length distribution, and span the observed distribution of GC-content across target regions. The spike-ins are distinguishable from cfDNA based on specified reference mismatches, the pattern of mismatches was chosen such that they are unlikely to be observed from natural processes. These spike-ins are used to calculate estimates of false negative rate across GC contexts and predicted hybrid capture overlap.

### Model Overview

The model identifies regions that are recurrently somatically mutated (focal amplifications, translocations, inversions, single nucleotide variants, insertions, deletions), and pre-specified loci (such as oncogene exons), and chooses the most informative combination of regions up to a specified total panel size.
Specify genome length, genomic alterations under consideration and force inclusion of specified genes.
Specify tumour variation database under consideration and tumour types, and relative weights of each database.
Specify if correct for population incidence of each tumour type (to guard against sampling bias.
Specify if the regions should be generated for exons, or at genomic level. (These regions are already corrected for uniqueness in the reference genome).

FIG. 6 provides a schematic representation of a hybrid capture panel design process, including data transformations. Drums represent databases, dotted boxes represent inputs, diamonds represent operations, and solid border boxes represent outputs.

### Region Selection Optimization

To identify the most informative regions of the genome to target for a specified total panel length, the signatures of genetic alterations recorded in cancer mutation databases such as COSMIC are assessed. The optimization is done using either Forward-Backward optimization or Greedy Optimization.

The design is then validated using a cross validation procedure to account for potential biases induced by constructing the panels from a limited number of samples. Cross validation strategies are important when designing cancer panels because the genetic variation in samples is heterogeneous both within tumours (intratumour heterogeneity) and between patients (intertumour heterogeneity), and are influenced by factors such as genetic background (e.g. POLE mutation status), environmental exposure (e.g. smoking history, previous therapy), and tumour stage. Therefore, the structure of the underlying population can influence the panel design, cross validation is a well-known strategy to guard against such structure.

### Forward Backward

Loci are identified by alternating between forward and backward passes until a panel of specified length is constructed from L loci. Loci are stratified into those included in the panel (chosen loci), and those not included on the panel (available loci).

Forward pass of iteration: Identify the locus in the available loci which adds the greatest number of somatic mutations to the panel, f*.

Backward: Include f* into the panel set, then identify the locus in the included loci that adds the least somatic recurrence, b*.

If f* does not equal b*, exclude b*. Start next iteration.

This scheme identifies the optimal set of loci for combined somatic recurrence. The optimization exits when the panel length is reached.

### Greedy Optimization

Start with the locus that adds the greatest somatic mutation load, add this to the panel, then choose from the remaining loci the locus with the greatest somatic mutation load. Terminate when the combined sequence meets the specified panel size. This algorithm is not guaranteed to identify a global optimum.

### Cross Fold Validation

Cross Fold Validation is used to assess the stability of the identified panel accounting for the influence of structure in the disease databases.

Construct two mutually exclusive sets of patient samples, with the cardinality of the sets determined by the training proportion p. Generate a panel on the first set that has cardinality p recording the total number of patients with mutations on the panel. Validate the proposed panel in the validation set that has cardinality (1−p), calculating the proportion of patients with mutations on the panel. If the patient proportions are within a threshold, T, retain the panel. Otherwise revise.

### Database Queries

To obtain samples of genetic variation databases of tumor biopsy sequencing are queried, which are stratified by a number of patient covariates such as disease type, stage, environmental exposures, histology, etc. All germ line genetic variants observed in population sequencing of healthy populations are removed, such as the 1000 Genomes database, to guard against false positive variants in the cancer databases which would confound the panel design (identifying mutations found in cancer, not in healthy individuals, is the goal). There are known germline mutations, such as BRCA1/2 mutations that predispose individuals to cancer, which might be eliminated through this approach. However, regions of interest are allowed to be forced into the design to incorporate this information.

### Data Transformations

To account for differential performance in hybridization capture, information about the sequence properties of the human genome are incorporated into the panel selection process.

Specifically, metrics about the uniqueness of each base in the genome are incorporated, since this drives the specificity of the hybrid capture. For example, if a locus is homologous (identical) to 99 other loci in the human genome (e.g. a LINE element), a capture probe would only pull down an average of 1 relevant locus per every 100. The metrics used are 1).

This information is incorporated by using two pre-calculated summary statistics of genome uniqueness available from the UCSC genome browser database.
1. Mappability, s, which quantifies the uniqueness of kmer sequence alignment to the genome

$$S = \frac{1}{( \# matches in the genome )}$$

2. Uniqueness, u, the uniqueness of 35 base windows across the genome in 1 base sliding windows

$$u ( x ) = \{ \begin{matrix}1 / x , & x < 4 \\ 0 , & x \geq 4\end{matrix} ,$$

where x is the number of exact shared sequences

Both maps are combined, and then a character encoded uniqueness value is generated for each base in the human reference genome. Hence, the reference genome is transformed from a sequence of nucleotides, to a sequence of nucleotides annotated by a hybridization specificity score f (s, u).

Software descriptions
createEndcodeReferenceGenomes.pl

### Inputs:

a BED file of the human reference genome (zero-based coordinates). Either

wgEncodeDukeMapabilityUniqueness35 bp.bed—How uniquely kmer sequences align to the reference genome, s, where s=1/((“# of matches in the genome”)), e.g. s=1 for one match in genome, s=0.5 for two matches.

wgEncodeCrgMapabilityAlign36mer.bed—how unique each sequence is on the positive strand starting at a particular base and of a particular length (here 36), u, where u=0 for >=4 matches, 0.25 for 4 matches, 0.33 for 3 matches, 0.5 for 2 matches, and 1 for unique match.

### Outputs:

A FASTA format file *.refGen. Each base in the genome encoded with character encoding of the reference genome uniqueness/mappability according to
“chr” (65+“int” (20*V))
where V is either, s or u as described in inputs.
explore[MUSIC,COSMIC]samplesIDs.pl

### Inputs:

*Uniq*.refGen from createEncodedReferenceGenomes.pl

*Map*.refGen from createEncodedReferenceGenomes.pl

variation databases

TCGA somatic_mafs_cleaned_filtered/*_cleaned_filtered.maf

COSMIC . . . .

### Params:

Ignore1000G <BOOL> exclude variants observed in 1000G
checkMappabilty <BOOL>
mappabilityThreshold <DOUBLE> threshold for bases >=threshold

### Outputs:

Exons.txt <gene-exon#, length [bp], gene, exon, chromosome, start, end>
Bins.txt <chromosome-start-stop, chromosome, start bp>
Mutations_inBins.txt <TCGA tumour-v-TCGA normal, chromosome-start-stop, mutation count>
Mutations.txt <TCGA tumour-v-TCGA normal, gene-exon#, count>
Kernel.txt <chromosome, postion, mutation count, mutation count*prevalence of disease>
Samples.txt <TCGA tumour-v-TCGA normal, disease type, mutation count>
allPositions_preQC.txt

### Operations:

Load 1000G data and exclude all sites from analysis
Exclude all loci that have reference Uniqueness <=mappabilityThreshold
Exclude all loci that have reference Mappability <=mappabilityThreshold
Generate bins across the genome in intervals of 200 bases
Exclude all samples with >=1000 Genome variants
Exclude all samples with >=tumour site
For COSMIC: exclude all TCGA samples, retain genome wide, non-coding, insertions.
Count up mutations per sample id, per exon, and per bin.

Aspects of the invention include methods for estimating sequencing errors for the calibration of variant frequency estimation. It has been observed that circulating tumor DNA (ctDNA) fraction is correlated with tumor size, stage, treatment response, and prognosis. Imaged tumor size is used to track treatment response and remission. It has been shown that tracking ctDNA variants has high correlation with imaged tumor diameter (>90%, Pearson correlation (other research has shown similar results using tracking tumor identified mutations). Hence, the accurate estimation of somatic mutations from ctDNA has the potential to inform clinical decision making for patients.

### Using Multiple Nucleotide Somatic Variants in a Patient

Here, a process is described to estimate the impact of sequencing error, and non-uniform coverage, on variant allele frequency estimates using somatic alterations in the sample. To do this, identify a somatic variant that has N consecutive bases differing from the germline genome (N>1), let this be represented by the vector V={a(1), a(2), . . . , a(n−1), a(n)}, where the elements a(i) represent the differing base at location i in the variant. Such variants can be generated by somatic alterations: translocation, inversion, insertion, deletion, amplification, or mutation. In some embodiments, one or more considered bases need not contain a somatic alteration, provided such considered bases are sufficiently close to one another (e.g., within about 1, 2, 3, 4, 5, 6, 7, 8, 9, 10, 11, 12, 13, 14, or 15 bases of one another).

For each base a(i) in the variant, count the total number of alleles supporting that base, this generates n estimates of the frequency of V, f(V). All the observed frequencies f(a(i)) should equal f(V), but due to variation in coverage and sequencing error, this may not be the case. Known statistical methods can then be used to quantify the dispersion in frequency estimates that arise during sequencing. This can then be used to correct frequency estimates. One example would be to use the sample mean and variance to estimate a confidence interval using an appropriate sampling distribution.

### Using Heterozygous Germ-Line Variants in the Patient

The ratio of alleles at heterozygous sites should be 1/2 in diploid organisms. There exist large databases of SNPs segregating in human populations. For a given individual, these sites can be interrogated and heterozygous sites identified as loci with two alleles with roughly equal allele frequencies. An empirical distribution of allele frequencies can then be constructed from the observed frequency of the second allele at the heterozygous sites. If the number of heterozygous sites is large enough, frequency estimates can be constructed per allele combination (A>C, A>G, . . . , T>G). The distribution can then be used to correct frequency estimates at the somatic variant sites.

### Spiking in DNA Controls

A known input amount of DNA, that has distinct sequence from the patient is added to the sample. These are positive controls for variant alleles in the sample.

To generate an identifiable spike in, sequences that are unlikely to be observed in the human population are generated. This is done by 1) choosing regions that have low reported diversity in population sequencing databases, 2) introducing changes to the sequence that do not reflect natural mutation processes (e.g. the sequence (same)n,{change, same, change, same, change},(same)n). The control sequence is further distinguished because the length of the spike-ins (120 bases) is known and so are the location of the introduced changes.

It is known that hybrid capture can be impacted by the number of mismatches between the capture probe and the target DNA. Four mutations were introduced into each control. The spike-ins were constructed so that the impact of 1) GC-content and 2) probe-target overlap can be observed by 1) choosing sequence with differing GC-percentages from the known GC-content distribution across the targeted regions and 2) varying the percent overlap of the 120 base long control DNA with its corresponding pull down probe.

The spike ins are added to the blood collection vacutainer before blood draw so that a) samples can be identified from their sequencing allowing the identification of sample mix-up in the sequencing, b) so that contamination from apoptosis of nucleated white blood cells can be estimated (this is described further herein), and c) so that false negatives can be detected.

Aspects of the invention include methods for detecting contamination of cell-free circulating DNA in humans. FIG. 7 provides a schematic overview of one method in accordance with embodiments of the invention.

Cell-free circulating DNA from human blood plasma (pDNA) contains, besides a majority proportion of molecules derived from a person's normal (typically healthy) genome, fragments of tumour DNA in cancer patients and fragments of fetal DNAs in pregnant women. Surveying that admixed portion of either tumour or fetal DNA is intrinsically challenging, for the admixture proportion of the cancer-/fetus-derived molecules can be as low as 1 in 5000 molecules.

Any given unprocessed blood sample, typically but not always stored in an EDTA tube or different type of blood collection vessel, will contain a certain fraction of cell-free DNA as well as white and red blood cells (WBCs and RBCs). After a period of time (and influenced by environmental factors such as temperature), the contained WBCs will undergo cell death and start releasing the contained DNA fragments into the circulation. Due to the process, any tumour- or fetus-derived cell-free DNA contained in the blood sample will be further diluted, rendering their detection and characterization even more challenging.

Technical solutions (e.g. Streck tubes) exist to prevent the contained WBCs from rupturing and releasing their DNA, but these solutions are not perfect and the problem of dilution remains, in particular if the blood is stored for longer periods of time or transported.

For any diagnostic method based on surveying the existence or characteristics of tumour- or fetus-derived DNA, it is thus desirable to measure and control for potential contamination.

Potential use cases include:
1. establishing a sample-specific lower limit of detection,
2. quality-controlling a sample, including the rejection of a sample with too high an amount of contamination or not producing a diagnostic readout for a sample with too much contamination,
3. re-scaling the detected number of aberrant/tumour-derived/fetus-derived molecules by estimated number of contaminant molecules,
4. using the re-scaled number of detected molecules as a more accurate representation of disease status or progression than the raw number of detected molecules would allow for.
5. estimation of the absolute amount of dilution.

In some embodiments, a methods comprises one or more of the following steps:
1. Identification of a stretch or a region in the human genome that is a) homozygous in the vast majority of the human population (or homozygous in the vast majority of the desired target population) and b) high in genomic complexity, i.e., establishing the genomic origin for molecules derived from that region is, using standard algorithmic methods for read alignment, unambiguous and unchallenging. Typically that stretch would vary in length between 50 and 150 bases, but the method described here can utilize both longer and shorter regions.
2. Perturbation of the sequence of the stretch or region by either substituting a number of nucleotides with different nucleotides or introducing or deleting a number of nucleotides. Typically this step would include the substitution of one or two nucleotides located centrally in the sequence with different nucleotides.
3. Verification that the so-perturbed sequence is not present in the normal human population. A variety of standard methods exist to achieve this, for example genome alignment or comparison to a de Bruijn graph generated from population sequencing data, such as the 1000 Genomes Project. If this verification fails, Steps 2 or 1 need to be repeated.
4. Biochemical synthesis of (approximately or exactly) n copies of the so-perturbed sequence, using DNA synthesis methods. Typically the number n is chosen so that, when the n molecules are introduced into the blood taken in Step 6, the ratio between n and the expected number of copies of the human genome in the volume of blood drawn resembles the expected/desired minimum ratio between tumour-/fetus-derived fragments and normal genomic fragments. (E.g. if one expects 1 in 1000 circulating fragments to be of tumour origin, and if one ml of blood typically contains about 1000 copies of the human genome, and one draws 5 ml of blood, n=5 per tube would be a sensible choice).
5. One of the following steps:
   a. Producing a blood collection vessel that contains the n synthetized copies of the so-perturbed sequence. This could be a standard vacuum tube, a vessel specifically designed to prevent WBCs from rupturing, or any other kind of blood collection vessel.
   b. Producing an additive component, containing the n synthetized copies of the so-perturbed sequence. The additive component will dissolve upon contact with blood and then release the n copies of perturbed sequence.
6. Filling the utilized blood collection vessel with human blood at time X. If Step 5.b was employed, addition of the additive component immediately after or before addition of the blood. Typically the blood vessel would now be transported to a processing facility, for example using a courier service.
7. Extraction of cell-free circulating DNA by centrifugation and DNA library preparation from the extracted DNA.
8. Measurement of the frequency of the perturbed sequence (f_{P}) and of the frequency of the unperturbed sequence (fₙ), using the technology that will be used in downstream interpretation of the sample. Typically the frequencies would be measured using a digital PCR-based approach or a sequencing-based approach, either utilizing a whole-genome sequencing method or a targeted sequencing approach.
9. Interpretation of the observed frequencies:
   a. f_{P}/(f_{P+}fₙ) is an estimator for the post-dilution frequency of tumour- or fetus-derived alleles originally (i.e. before dilution due to rupturing WBCs started) present at n copies in the sample.
   b. Depending on the properties of employed downstream interpretation technologies, if f_{P}/(f_{P+}fₙ) is 0 or small, the sample should be rejected or not be interpreted.
   c. Multiplying the observed frequency of an assumed tumour- or fetus-derived allele in post-dilution data by [(f_{P+}fₙ)/f_{P}]×n will give an estimate of the pre-dilution absolute count of that allele. Tumour allele counts and their development over time have been demonstrated to be important indicators of disease status and progression.

Carrying out the above procedure for different genomic loci and different values of n confers important additional advantages, including but not limited to, the following:
Utilizing different genomic loci will reduce statistical variability and can, if the loci are selected from a range of local sequence contexts, be used to control for GC content bias.
Using different values of n will enable the (more accurate) estimation of the total amount of dilution (measured in dilution-derived molecule fragments) and hence the pre-dilution number of DNA fragments in the blood sample.

To estimate the number of contamination molecules, denoted by c (i.e., those DNA molecules released from apoptotic nucleated cells in the blood sample), a two-step sampling approach can be used. Note that c monotonically increases with time. The perturbed sequence (as identified and synthesized above) is referred to as a benchmark sequence. Let the number of sampled pDNA molecules at that position in the genome be denoted by d.

At the blood collection, b1 is added immediately to the sample (indeed the collection vessel might even be pre-seeded with the first benchmark sequence molecules, see above). Thus the frequency of the first benchmark is

f₁=b₁/(d+c(t=0))

The sample is then transported to a collection facility. Preceding pDNA isolation from the sample at time T, a second measurement of the frequency of the benchmark sequence is taken. The sample frequencies f(1) and f(2) are observed, the difference in the observed frequencies is then calculated to determine the number of contaminating molecules.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method for sequencing a nucleic acid, the method comprising:
obtaining a plurality of sequencing reads of a nucleic acid in a sample;
identifying an ensemble comprising two or more sequencing reads with shared start coordinates and read lengths;
determining a number of original input molecules present in the sample that correspond to the ensemble sequencing reads;
identifying a candidate variant in the ensemble; and
determining a likelihood of the candidate variant being a true variant using a probabilistic model and the determined number of original input molecules.

2. The method of claim 1, wherein obtaining the plurality of sequencing reads comprises:
preparing a sequencing library from the sample;
amplifying the sequencing library; and
sequencing the sequencing library using next generation sequencing (NGS).

3. The method of claim 2, wherein preparing the sequencing library comprises ligating adapters to the nucleic acid at a temperature of about 16 degrees Celsius using a reaction time of about 16 hours.

4. The method of claim 2, wherein amplifying the sequencing library comprises PCR amplification, and wherein the method further comprises selecting an over-amplification factor and a PCR cycle number required to detect variants at a specified concentration in a sample using an in-silico model.

5. The method of claim 2, further comprising:
designing a hybrid capture panel to target a genomic region based on one or more factors comprising: guanine-cytosine (GC) content, mutation frequency in a target population, and sequence uniqueness; and
capturing the amplified nucleic acid using the hybrid capture panel before the sequencing step.

6. The method of claim 5, wherein capturing the amplified nucleic acid comprises using a first hybrid capture panel targeting a sense strand of a target loci and a second hybrid capture panel targeting an antisense strand of the target loci.

7. The method of claim 2, further comprising adding a synthetic nucleic acid control to the sample before amplification of the sequencing library and determining an error rate using sequencing reads of the synthetic nucleic acid control.

8. The method of claim 7, wherein the synthetic nucleic acid control comprises a known sequence having a low diversity across a species from which the nucleic acid is derived, and having a plurality of non-naturally occurring mismatches to the known sequence.

9. (canceled)

10. The method of claim 7, wherein the synthetic nucleic acid control comprises a guanine-cytosine (GC) content distribution that is representative of the target loci of the hybrid capture panel.

11. The method of claim 7, wherein the synthetic nucleic acid control comprises a plurality of nucleic acids comprising varying overlaps with a pull down probe of the hybrid capture panel.

12. The method of claim 7, further comprising determining an error rate using sequencing reads of the synthetic nucleic acid control.

13. The method of claim 7, further comprising determining a candidate variant frequency.

14. The method of claim 1, wherein the nucleic acid comprises a cell free nucleic acid.

15. The method of claim 2, wherein the sample comprises a tissue sample, and wherein obtaining the sequencing reads further comprises fragmenting the nucleic acid before preparing the sequencing library.

16. (canceled)

17. The method of claim 1, further comprising applying a deterministic model to the candidate variant before applying the probabilistic model.

18. The method of claim 17, wherein the deterministic model comprises discarding the candidate variant when the candidate variant is not identified on both a sense and an antisense strand of the nucleic acid.

19. The method of claim 1, wherein the probabilistic model is a likelihood estimation model.

20. A system for identifying a nucleic acid variant, the system comprising a processor coupled to a tangible, non-transient memory storing instructions that when executed by the processor cause the system to:
identify an ensemble comprising two or more sequencing reads of nucleic acids from a sample, said sequencing reads having shared start coordinates and read lengths;
determine a number of original input molecules present in the sample that correspond to the ensemble sequencing reads;
identify a candidate variant in the ensemble; and
determine a likelihood of the candidate variant being a true variant using a probabilistic model and the determined number of original input molecules.

21. The system of claim 20, further operable to apply a deterministic model to the candidate variant before applying the probabilistic model.

22. The system of claim 21, wherein the deterministic model comprises discarding the candidate variant if the candidate variant is not identified on both a sense and an antisense strand of the nucleic acid.

23. The system of claim 20, further operable to determine a target genomic region for the two or more sequencing reads based on factors comprising: guanine-cytosine (GC) content, mutation frequency in a target population, and sequence uniqueness.

24. The system of claim 20, wherein the probabilistic model is a likelihood estimation model.
